SEQUENCE LISTING (12 sequences)

SEQ ID NO: 1 - DNA, 2874 bp, Homo sapiens
ccagcgaggatgcagacgagttgcacaaaattttactggagaaaaaggatgcctgaacacgcaaagtcggctgcagaattattgccaagttgctgctgcttccaccgccccttagtcagtttttcttctcttctttgacattctaagaacttatagataacttaaaacttttgtgaggaagattaatgtggccaataaaacctttaaatgttaagtgtcaagaaactgcactctcccttcttaagaactgcctaaagtgtaaaatacatttgaatgcaatttttggaagattttttaatgttcgtttattaaactaaccctaagtgatttcttcaaggactgcaatcagggtatcaatttgctttcccaaaggctcttccaacccgtgggttttggggtccaccgccaccaccagagaggcttttgaacaggtgcctggctgtgttcagaaggaagctggcctgtgtgcttctctccggtgggctcagccgacgtgtgagacttgttctgttaccaaatgaaccgggctgccacgctgtgacaggcgtttgtcctctgctttatttttactttgaagctcaaatgcgagtactaagtgttcacctcagcgttcgaatcatgtaaccctgtgggctgcttcacgagaattcaggacctgcattttcattctaaaaagaaatgaacagcttgtgaaggagttttttggcttcatagtttctattcatgaggtagtgttacttctttatccccctaaagacaaaatgaagataaagggggattgccaggaatgggtttaaaagcacaaatgtggtagcttatcatctacaccatggagagtgaacccttacgaaatgaaagtcaaatgagaccatccgagaaaaagatgcgcataggcatttgtaccatgatcaaccccacgcacatgaaaactgtgaccaagtgacgtgcctgggagctttgacacacgagccgtgtgaattcactaggaaacatgtaataaagtcatggaagagaaaatcgtgtgtaaattttgcctttaactttagaccgcagtatattataatacatttgatatctgaaatatctttacttttttaagagtaagattccatatgtctgtctggaagggagccatggttattcacacgaatatccctgtcacttctccagaggtgtcaggtaactaacacgagcattctttgaagactctgggcacatgaatgatacacagaattgaatgtttaaatttccactttgagtcctcatgaatcatttgagactagtaccagctgatcttgtgtacaggctcagggtcagtgcccaagggctcccgcgtgtgtgttctgatcttcagtgcgtagcacattctccatttagaaaagagtggtcagaataattgtggacggtacagtggctttttaaaactacagtctttaggtgtaaggtttggcgccgggagcaattttatgatcaaatatgatgaactcctaagtcactgaggtgtgattgggccaatgttggcatgaggttcttgctctacttccagtgttttgattccactgggagaatttggcctagtgtgtggctttggatgaatccgtgtagagagaggtgagcttgtcctgttacagatgctgtcagacatagcgatagtaggcacctagggaggaagtggccgttagttttacactgactttttaagaatggagaatgcacgtgggtttctgttgcggatgattcatagtaagcaagcggttgatgctgttaataccggccccacccgattgacattaagtttattcagcttttaaaawgatgaagaactarggggaacaaatttaagtttgttgcaacttagccacacatgcttccctggtaccagctggaatcagcagctcacaggcatcttcaggacacttcagtgtatatgacacagtactttgttagcgtctgcgtgtgtatggaaagttgacaaaaaatggcatgaaaagatcatgattggattttcttttaaacctgcccttctgtaaaaaatagtttatatatttttaaattagtaggtatgtgtggcttccttttttcctaacattcccagcaaatttttgctgctaagactatcactgttaaagtgaaaattacagggaaaaatgtgatgaatataccgtaactcaaaatgtgatattttcttaaaatcactcttttatgctttaggaactggttggtctccactttgattattagtgtaaagagcctgagtatacgtggatttcattgtaaaatttaactccttgtcttttacttggggcacggggcccctggagggcttccctactttccccactatgttaacaggtaattctgatttatgcgtttagtttgacttatttttaacaaaatattagaagttatgctttaaaatgtttaatgtggactgaaattttcatcttttgtttgagaatctatgaagtgtatcatatacgtggcctaaagcaaggtgtgtattttgttattctgaaattgttttgcatctggacaaatactaaatatcccagtggccttttttttttttttttttaaacctgtgtatccatctcatccttttgcgcattcctagtaagcaaaaaaatttgttatgccatcttcattattcgaattacagactgaaaaaatatggccagtttttaaagaagtttagattatgttttccatggaaggacaagtctgactgttcataggctgattttctttaagaggattattctgttttacaatttcaattctagatcacattttatatatgctgcatgccaaaaaaaaaaaaaaaaaaaaaaaaaaaaaaaaaaaaaaaaaaaaaaaaaaaaaaaaaaaaaaaaa

SEQ ID NO: 2 - DNA, 1859 bp, Homo sapiens
gagcagactattgctgatatgttggttttaattcaaaagaaacgatgatgccaaatggtttggaattgacagcagctaaggaggaaaaaaaaaaaaaaaaagaaagaaaaaaagaaaaggctggtgacacccccctggcgtggttgacctggtcccagtcgcagtccctatggcagcaggcacgcgtaaaacagaacctgatgacggctgaaaatcaagccagactgctgtgcggctagcttgccgccgtggaatgccttgtgtcttggccatgtccagccaagggtacaacgtgcttgttcccgatcacccaggtttgccattggaagtcaaagacagaatcgcttcatggcactacagatgtggaaaaataaaaatctcagctagaaagaacgtccgatttggagatagcgggaggacacgaaggagtgggggccattttggtgctgagaggagggtgccccaacttcaggaggaccatgtgacggctgtggttgttttcggggtcacttgcagcacacacagcgtccccttgatgctcgatggggaccgcagacgggctgcagacctaacccctggctgtggacagagaggctgctccaggcttctttccttctcaatgctttacacaggatttccttcatttgtgctttcctttggtttaacagttaaaaaagaagagtgagggggcaaaatggtttgtcacttgtccaaaactgagagaagaggtggaagtgggcgccaaatctcctgggtgatgcttcctggtcctggcgatcggttgcttgctcagggtttgggagctgttgctctggaaccaccgctggccttctgggacctcgcttccgtggtaggggacgtgaaccagcctcctggtggagctttgtgttgcagtgaggccacaaagcaaaggcccaggagcagaggcctgacactggctgtggtcgacggtcacaccttgactccctctctctctctgaatatacaacgtgtgggtgggcccgttcagcagatgttacaggaaaaatagcaaatttttaacttattccatctccaaagttgaaaaagatcagacagttactaaaataaacgatttctcaattgcattctggtgccgkggcccggtggccgcggcgtgggcggggcgttggaggtgggagggccccggctgagtgaggggctcactcaraacaggcacagtcagctgggctgagcgaggctcaragtaaggcggtgttcctcacagaagaacacatcggaaaaagctgctcctcttctgctggtccggtgtgattttgactccctggttgctccctggggctgttgccttccattttttgtccatttttgctttgatatctctggcgagagtgaaaaatgcattttccacattgatgttggccttcgcgctggtctccatgaacttgattccatagtcgagggccagcttttctccccgttccttggaaacttgtctcttgtcattcacatcacacttgttcccaagtatcatcttttcgacgtctgcagaggcgtgctcctcaatgttgcgaatccagttccggatgttgtcgaaggacttctcgttggtgatgtcgtagaccagcatgatgcccattgcacccctgtagtaggccgttgtgatcgtccgaaaccgttcctgaccggctgtgtcccatatctgcagtttaattctcttgccatcgagctctatggkcctaatttaaagtcaattcctatggtggagataaaagtkgagttgaagcgtcctysgagaagcggaacaggacacaggtcttycccaccccskagtccccgatcagcagcagcttgaacaggtaatcgtaggtcttcgccat

SEQ ID NO: 3 - DNA, 5577 bp, Homo sapiens
ggccctaacaggaatgaaactgaggtgtcaagagggctctctgtgcacacttttggccatgacccagtgtcttctgcagtccttacgcagccacatgaggacactcagcacagagcagcctgtgtgtcccagagagtgagagaactgaagtggtgtccccaaggccacccggcaagttggtggcagagccaatacctgagctacccttaggccccgtatgtacctgcttctcatgtgacgcacagggaaattgaggcctggccccacctccctctgttgccctgctggccacatgcccaggggaagggatttccagggcttacccagagtggcattgctggggagagaccagatgcctgggctcctgggtttccccaaggggacggcccttaggaatcctgtgcctcctccactgccacccccttcacagcgggtcatccggagccgcagccagtccatggatgccatggggctgagcaacaagaagcccaacaccgtgtccaccagccacagcgggagcttcgcgcccaacaaccccgacctggccaaggcggctggaatagtgagtgccctccccctccccaggccccggcccctccctggggggccctcagctctcctctgcctcctgaggcctgactccaactctctctgttgccctgctgcccacatgcccatcctaggctctggatttggtctagccactactttccatgggaggggggtgaagtgcccaggccaggacactgcggtgctgacagcttgcagcctgcagccccttcccaagctccttggccctcccctcctcctggccctttatgcattgaggtgtgacttcctgcaggtcagccctgggacagcctctgtgtctcattccttattgagtatctatctgtttgctggggactgggctgctgtgtgggcacctactgtcaagccttgggtttctgggagcacctactgtgtgtcgggctgtgggcacccactgtgtgtcaggccctgggctgctgtgtggacatgcactctgtgagcatctactgtgggcctggccctgagtacctgtgagcacccactgtgtgtcaggtcctgggctgctgtgtgggcatcaactctgtgagcgcctactgtgtgcgcagccctgggctgctgtgtgagaacctactgtgtgtcagaaaatggactgctatgtgagcacatcgtgtgtgataagccctagatgtcagtgaacacctactgtgtgtcaggaagtgagctgttgtgtgggtactttctctgtgagcacctcctactgtgtgtccgcagcagccagggcctctgtggtgtggctgcctattgtgtgtcgggaatctggcttctgtgtgagcatctccagagggagcacctcctgtgtgatcactgactgttgtccaggttctgggattctgctgctcacactcaggagtgctgggcacatggatgaatacggcctatggctgtgggcctcactgctgtccactgcctagtggccaccccaggacactgcacccactgctgtgctccccacccatccatccagccacccatccatccacccacccatccatccatccacccacccatccacccatctagctatccacccacccacccatccacccacctacccatctacccatccacccacccacccactcatccatccacccacccatccacccacccatccacccatcaatccatccatccacccacccatccacccatccatccatccatccatccatccatccatccatccatccatccatctctatccatccctctatctccatccatccatccacccacccaccgatccatccatccatccatcctttattgactgtctactgcatgccaagccctgcgctcagtgctgtgaggctatgtcacgtggcaggagggaattccccgacctctgctgtccagcagtcaccaagcacactgtcatgcgaggagcaggcccaacctcccccaacccagttttatcaaccactcccttcctctccaccagagtgacccagcctcctgtgggtggccgagaggggccccagggacaggaccaggccagcagcacccaccatggcagtaaccggacccatttctgttttgtttttgcacaactctccctctctctgtgtttcctcctttcattctactctctctccttgtttttttgttccctcctcccttccctttccccgtcctgtggcctctctgccctttggctctctgtttctccttccttcttgcacctgtctttttttgctccctctcctccttcccttctccgctcttctcaccctcgctttttcctttcggccttcccctggcttccttctctgtctctgaccctccctgggcctgtgtctgtgtccttgcgtccctgtctctctggatttccctctgtgcccatctggtgtgccctcgctggctcacgcgtgtccctgtctccgggtaactgtctgtccatctctcccccgtctcccttgtcctctgtctctccttgtgcttctcgccttcctttccccggccctatctctcccattgcctcctgcaccgttctcttcctttttctgtctgtcttcctcctttccctgcctctcctcctctctgtgtctccctcccaccatctctcttgctctctgactctctctctgtctctctctctctgcccccgccctctgctgcttgccagtcattgcttattcctgggaaaagtgcgagtagattcggacgccggggcagtgccataggcataggaaccgtggaagaggttgtcgtccgcggggccgccggctctggaggctgcctgcacgcgctgttctctgctcgctctcaggacggaggccatattggggacgttgcccctctgcccccgggacaggccccagggcgtgcgggatggagtggcggcagctccgatggcactcagcacctgctttggggcctggtacctgtgccagagccagtgcccctcaccaggggcttctggccctgccttggcccctgggaccctggcccagtccctgccaggatccggtacccaaaggccctacacccagccgcacgtcccccaatatcgccggtctgcatggagcgccatccctctcctctgccctgactcctcctccccgccacgaagtgacctggggtcctaccccttcctgcctccagagaagctgggggcgggcttctggggcctggggcatcccagcacagtgtgtgggaagctgggggagtcttccagctgctgggccagaacctcccccagccaatttggaggttccggggaggggccctagctggcacggggtgggacttgggttgctacactgccctctgaccactgcccttaggctgcagatcccacagagccctggggggcggggagcggtagccattctgaggactcggcctcctcccaccctagccccctcaggatgctgtcctaatcctgggccagtattgacgtgcagtcctgccgtgtgagctcgggagagccccttgccctcctggggactgtttccctttcgtaaactgggaggctgtctctgggaatggatatcttctctcgttctttcttgctcatcaactctgcttcagggcctggggcagcaggatccccagaggggatgtgggggggcactggggctcccaggtaaggtggcactgagttggggcctctccccacagtcactgattgtccctgggaagagccccacgaggaagaagtcgggcccgttcggctcccgccgcagcagcgccattggcatcgagaacatacaggaggtgcaggagaagaggtgggtgagtgggggacagtgccccattcccctgcacccccatccctgagccccattcggtggcaaagcaaggcaggcagaagggagtgcccgcccctctgcctctccatccccactagtgacagctgtgtggtcaagtccctgctgcgtgtcgggcaccagggccagcacgtcacctaacgtgccacatgcagatcaagaagcggtaggtcagaggagtcaaggggcttgcccagatcacacagccagtgatgggcagagctaggatctgagccctgatctgtctagggccagcatccgtgctcttcccacggccccagcgcatgtgggagggcctagtgctggttttcagggtggccacagatgggcctggggggtccatgagtgtgggcagcatgagggcagtgagcccaggccagcagcagggctgcccccggacatcagaggctagctcccggctgcctccccgatattaaccatgtgtgaccttgggcgagtcactgacctcctctgagccttactgtcccaacctggaaaagggacaagaacacaacccatggcatggggctgctgtggggactcagggcactgcgtgtgaggccagggccaggtcccctgagagggctccaggacgggagtgggcattgtccttgctgctgccagagctccgcatgctgtgagtcctgggttcaggtcttggcactgccacgtcctagctgtgcgtccctgggcaggttccttatccataatgggacagctatacctgctcctgtggagaggacctgggaggagtcccatcctgtcccatatagccccatcagtgccaaccccatcactggccaggctagccagggagcccacaagactgctccagggctggccctgagtataggggcgtgggtatggggcaggaggcaccgtgactcccctcaccgcctgggcctcacgctacgcctgatgccaggcctggtgctgaatccccccgctgcccccgtgtgccccgcagggagagccctccggctggtcagaagaccccagacagcgggcacgtctcacaggagcccaagtcggagaactcatccactcagagctccccagagatgcccacgaccaagaacaggttggggctcagggcacgtggggcttgggggcttgggagtggtgaaccgtccttcccctcccctgcctgggcccgggacagcacaggagccttgactstgccacagcagggtgtcagggggacctgggcattctctggggccctcctttgacatatacccagcgagcactttgtcacgcccagccccgcgcccgrctctggagcacagaggtgcctgcgcataggtccccgctcacggcgcagtccatcagaacgcggctcataggtgttgcggatcatggtgataggaggaacccgaagcagggttgggggratgaagarggactgggggcagaggtgttttagatgsgtcctcggggagrcacctccatggggtgacatttgaattgggaactgaaccagccctgctaggactggcatggcagaggagctcggccagtgcagaggcctggggcacactccagggacagaaagaagggtggctgggagtggtgacgtatgcctggagtccacctacctgggaggctgaagcaggaggatgattttagccaggagttggagctgcagtgagctatgatcatgctgtaatagcactgcactctagctggacatcatagcaagactccattgctc

SEQ ID NO: 4 - DNA, 18 bp, Artificial sequence
  Primer
ccccccagggtatcacca

SEQ ID NO: 5 - DNA, 17 bp, Artificial sequence
  Primer
ccccggtccatcccttt

SEQ ID NO: 6 - DNA, 24 bp, Artificial sequence
  Probe
aaatgccgcatcactcgggacaat

SEQ ID NO: 7 - DNA, 19 bp, Artificial sequence
  Primer
tgatccagacagccgacca

SEQ ID NO: 8 - DNA, 20 bp, Artificial sequence
  Primer
cccatgatgaatttggcacc

SEQ ID NO: 9 - DNA, 24 bp, Artificial sequence
  Probe
aaatgccgcatcactcgggacaat

SEQ ID NO: 10 - DNA, 17 bp, Artificial sequence
  Primer
ggctgcttcgacctggc

SEQ ID NO: 11 - DNA, 21 bp, Artificial sequence
  Primer
aagcgagggtacgagtccttt

SEQ ID NO: 12 - DNA, 25 bp, Artificial sequence
  Probe
agaagcgcatcttcgggctcatggt

(19) United States
(12) Patent Application Publication
(10) Pub. No.: US 2006/0051753 A1
(43) Pub. Date: Mar. 9, 2006

(54) Methods for determining the response of cells to vegf and uses thereof

(76) Inventors: Stephen David Charnock-Jones, Cambridgeshire (GB); Stephen Kevin Smith, Cambridgeshire (GB); Cristin Gregor Print, Cambridgeshire (GB)

Correspondence Address: NIXON & VANDERHYE, PC, 901 NORTH GLEBE ROAD, 11TH FLOOR, ARLINGTON, VA, 22203, US

(21) Appl. No.: 10/504,077

(22) PCT Filed: Feb. 7, 2003
(86) PCT No.: PCT/GB03/00534

§ 371 Date: Apr. 4, 2005

(30) Foreign Application Priority Data
Feb. 7, 2002 (GB) 0202881.9

(51) Int. Cl. C12Q 1/68

(52) U.S. Cl. 435/6

(57) ABSTRACT

The present invention provides methods of monitoring the progression of a disease condition associated with angiogenesis or vassculogenesis in a human subject in which a quantitative determination of the transcript level of at least one gene shown in Table 1 (by which is meant one or more of any of Tables 1a to 1f) in a sample comprising cells obtained from the site of said disease is made, and compared with the transcript level of at least one gene obtained from a control sample of cells. The transcripts of Table 1 are found to response to VEGF in a statistically significant manner under a variety of different conditions, including following serum withdrawal. The invention also provides gene chip arrays consisting of all or some of the transcripts together with appropriate controls which can be used in the methods described.

### FIELD OF THE INVENTION

The present invention relates to gene expression profiles of endothelial cells in response to VEGF, and the use of the profiles in diagnosis and therapy.

### BACKGROUND TO THE INVENTION

Angiogenesis, the process by which new capillaries develop from pre-existing vessels, plays a major role in physiological as well as pathological conditions. The development of a new capillary network is a complex process involving basement membrane degradation and extracellular matrix proteolysis, accompanied by the proliferation and migration of endothelial cells, formation of rudimentary vascular structures and remoulding of the extracellular matrix. The regulation of angiogenesis is thought to occur via a balance between angiogenic inducers and inhibitors many of which interact with specific receptors on target cells. Several factors of both peptide and non-peptide nature have been shown to induce angiogenesis in vivo: epidermal growth factor (EGF), transforming growth factor-alpha (TGFα) and transforming growth factor-beta (TGFβ), tumour necrosis factor-alpha (TNFα, in vivo), angiogenin, acidic and basic fibroblast growth factor (aFGF/bFGF), vascular endothelial growth factor (VEGF), PGE₂ and monobutyrin. Inhibitors of angiogenesis have been identified ranging from complex steroids to polypeptides including thrombospondin, platelet factor IV, TNF-α (in vitro), TGF-β, interferons, angiostatin, integrin inhibitors, 16-kD prolactin.

Endothelium is generally quiescent in the healthy adult organism. A marked exception is the female reproductive tract, where the need for additional vasculature is constantly imposed by the periodic evolution of transient structures and by the cyclic repair of damaged tissues. Widespread and profound disruption of the female reproductive pathways were recently described (Klauber N, et al 1997 Nature Medicine No. 4 443-446) in mice treated with the angiogenesis inhibitor AGM-1470. These also showed that ovarian and endometrial cyclicity could be abolished rendering the animals infertile and that decidualisation and placentation were also disrupted by the systematic blockade of angiogenesis. It is most likely that the cyclic angiogenic events in the female reproductive system are coordinated by hormones, the actions of which may be mediated by angiogenic factors that are either directly or indirectly hormone inducible. Ovarian, uterine, and placental tissues have been shown to contain and produce angiogenic and anti-angiogenic factors. Among those various angiogenic factors, VEGF possesses several unique attributes which suggest it plays an important role in these tissues. Specifically it promotes mitogenesis of vascular endothelial cells, vascular permeability and it also modulates production of a number of proteolytic enzymes involved in the process of neovascularization. Thus it is able to regulate all the steps of neovascularization and is likely to be important in physiological and pathological angiogenesis in the female reproductive tract and other tissues. VEGF binding sites are detected in many adult tissues, indicating that VEGF is probably important not only in angiogenesis, but also in the maintenance of existing vessels.

The pivotal role of VEGF in the development of the vascular system is further emphasized by the recent data (reviewed recently by Risau (1997, Nature 386 671-674). Loss of a single VEGF allele leads to embryonic lethality which indicates that even a relatively modest reduction in VEGF level can have profound effects. Gene knockout studies have also demonstrated that Flt-1 and KDR (the receptors for VEGF) are essential for the development and differentiation of embryonic vasculature. Mice null for the Flk-1 gene lacked vasculogenesis and blood island formation, resulting in death in utero between days 8.5 and 9.5. Mouse embryos homozygous for a targeted mutation in the Flt-1 locus died in utero at mid-somite stages.

Vascular endothelial growth factor (VEGF) is a heparin binding, secreted homodimeric glycoprotein of 30-46 kDa, also known as vascular permeability factor. It is a potent mitogen for vascular endothelium, possesses potent vascular permeability-enhancing activity and modulates the expression of several proteolytic enzymes involved in angiogenesis and also has a role in the maintenance of newly-formed blood capillaries.

Analysis of the VEGF gene has revealed that ‘the protein coding regions’ are arranged in eight exons. By alternative splicing of the exons five different mRNAs for VEGF are generated, which have 121, 145, 165, 189 and 206 amino acids respectively (VEGF₁₂₁, VEGF₁₄₅, VEGF₁₆₅, VEGF₁₈₉, VEGF₂₀₆). In most tissues the 121 and 165 amino acid forms predominate and the 145 amino acid form is generally the rarest. This form was initially described in human endometrial and placental tissue (Charnock-Jones D S, et al 1993 Biology of Reproduction 48:1120-1128) and has recently been shown to have unique features not shared by other forms of VEGF (Poltorak Z, et al 1997 Journal of Biological Chemistry USA, 7151-7158). Rodent and bovine VEGFs are predicted to be one amino acid shorter but are generally highly conserved. Recently several other proteins have been identified which show considerable homology with VEGF. These have been termed placental growth factor (PLGF) (Maglione D, et al 1993 Oncogene 8 925-931), VEGFB (Olofsson B, et al Proc. Natl. Acad. Sci. USA 93:2576-2581), VEGFC (Joukov V, et al 1996 EMBO Journal 15:290-298) and VEGFD (Yamada Y et al 1997 Genomics 42 483-488). It has been shown that placental growth factor can form heterodimers with VEGF and that these heterodimers can bind to one of the VEGF receptors. However, they are 20-50 fold less mitogenic than VEGF 165 homodimers.

VEGF acts through two tyrosine kinase family receptors which are c-fms-like tyrosine kinase (flt-1) and the kinase domain insert containing receptor (KDR). Both flt-1 and KDR possess seven immunoglobulin (IG)-like loops in their extracellular domains, which are different from the previously described class III receptor tyrosine kinases which have five. They also contain a single transmembrane region, and a consensus tyrosine kinase sequence which is interrupted by a kinase-insert region. The second IG-like extracellular domain of Flt-1 is essential for ligand binding and specificity. Both receptors have been shown to bind VEGF with high affinity. Flt-1 has the highest affinity for VEGF, with a Kd of 10-20 pM and KDR has a lower Kd of 100-125 pM. The murine homologue of KDR, fetal liver kinase-1 (Flk-1) has also been identified and shares 85% sequence identity with human KDR. Both Flt-1 and KDR/Flk-1 mRNAs are predominantly expressed in vascular endothelial cells in both fetal and adult tissues. They are also found on non-endothelial cells including peripheral blood monocytes, malignant melanoma cell lines, trophoblast-like choriocarcinoma cell line BeWo, and peritoneal fluid macrophages. Flt-4 tyrosine kinase receptor is related to the VEGF receptors, flt-1 and KDR, but does not bind VEGF and its expression is restricted mainly to lymphatic endothelia during development. mRNAs for flt-1, KDR/Flk-1 and flt-4 have distinct expression patterns and certain endothelia lack one or two of the three receptor mRNAs, suggesting that the receptor tyrosine kinases encoded by this gene family may have different functions in the regulation of the growth/differentiation of blood vessels.

The blood vessels that supply most adult tissues are stable, and their endothelial cells are quiescent and resistant to apoptosis. However, during tissue remodelling, blood vessels become plastic and are themselves remodelled to meet the changing requirements of the tissues they supply. This is most obvious during tumour regression and during the monthly atrophy that occurs within female reproductive organs. An important component of this vascular remodelling is endothelial cell apoptosis.

The withdrawal of survival signals may potentiate endothelial cell apoptosis during vascular remodelling. In vitro, endothelial cell apoptosis is induced by the withdrawal of fibroblast growth factor (FGF)-I, FGF-II, Vascular Endothelial Growth Factor (VEGF)-A or Angiopoietin (Ang)-1. In vivo, the treatment of human prostate tumours by androgen ablation therapy results in decreased production of VEGF-A by prostate glandular epithelium, which in turn causes the selective apoptosis of endothelial cells within newly formed tumour vessels. Importantly, in these tumours, survival factor withdrawal-mediated endothelial cell apoptosis precedes the apoptosis of the neoplastic cells themselves, and loss of tumor vessels precedes the decrease in tumor size. Other processes where the withdrawal of survival signals probably drives endothelial cell apoptosis during vascular remodeling include mammary gland involution, formation of the placenta and cyclical regression of the corpus luteum in the ovary.

The regulation of transcript abundance may supplement well-characterised post-translational pathways to orchestrate the apoptotic program in endothelial cells following survival factor withdrawal. For example, activity of the transcription factor p53 is induced by several pro-apoptotic stimuli, and many of the most important regulators of apoptosis are p53 target genes, such as p21/WAF-1, 14-3-3, Bax, Fas, DR5, PIG3 and Tsp1. Differential display and gene array experiments have identified transcripts encoding apoptotic regulators and machinery that are induced by p53. Another transcription factor known to regulate endothelial gene expression during apoptosis is NFkB. In healthy endothelial cells, NFkB-activated transcription of anti-apoptotic genes such as TRAF-1, TRAF-2, IAP-1 and IAP-2 is essential for cell survival. Endothelial NFkB activity is increased when apoptosis is induced by lipopolysaccharide, tumour Necrosis Factor (TNF)-α and etoposide. However, the role played by NFkB during endothelial apoptosis may be complex, since caspase-mediated cleavage of xIAP during apoptosis potentially reduces NFkB activity, and since NFkB can promote expression of both protective and pro-inflammatory genes in endothelial cells. Other transcription factors such as the E2F and Myc families could also play a role in survival factor withdrawal-induced endothelial cell apoptosis.

### DISCLOSURE OF THE INVENTION

The specialised nature of endothelial cells and their regulation by VEGF-A is essential for life. In part, their specialisation depends upon endothelial-specific combinations of post-translational signalling cascades as described above. However, this ultimately depends upon a distinct RNA transcript population i.e. the endothelial cell transcriptome and its regulation.

To investigate this, we analysed gene expression in a number of different contexts. Firstly, we combined Affymetrix gene array expression data with SAGE data to determine which transcripts were most abundant in human umbilical vein endothelial cells (HUVEC). Secondly, we compared the relative transcript abundance in HUVEC and other cell/tissue types, to determine which transcripts were endothelial-specific.

In two additional experiments, we used Affymetrix array hybridisation to identify changes in transcript abundance that occurred either when HUVEC were induced by VEGF-A to survive and proliferate following serum withdrawal, or when HUVECs in normal culture medium were stimulated by the addition of VEGF. During this study, we also found that primary endothelial cultures derived from different individuals displayed substantial transcriptome heterogeneity. Based on this finding, we suggest that genomics studies that employ single possibly idiosyncratic primary cell cultures may be misleading.

In summary, in the present invention, we have used a novel methodology to identify genes whose transcript levels are modified in response to VEGF-A in endothelial cells.

While other investigators in the prior art have identified various genes whose activity is believed to be modified in response to this factor, the methodology used by the present inventors differs in several significant respects. These included the use of primary cell cultures; the use of five independent samples, and the use of serum starvation prior to addition of VEGF-A. This latter step in particular was used to initiate apoptosis in a proportion of the cells, mimicking what would be expected in situations where, for example, a treatment of a tumour leads to tumour regression. Addition of VEGF-A leads to modulation of cellular transcript level. Using strict statistical criteria we identified genes whose transcript level was modulated significantly at 4 and 24 hours after addition of VEGF-A. Surprisingly, we found that at these two time points the transcripts identified at 4 hours and the transcripts identified at 24 hours had only 2 transcripts in common.

We have also used serum withdrawal on HUVECs for 48 hours to stress cells. We have identified changes which are robust and reproducible and are good pointers to the global and specific changes that occur when endothelial cell fate is perturbed.

Thus the invention provides a means to analyse endothelial cell fate in a manner which allows monitoring of a number of disease states in a useful and new manner. The knowledge of a number of transcripts, both of genes known as such and from ESTs, provides novel assay targets and allows the development of new therapies for disease.

While not wishing to be bound by any one theory, it is believed that the transcripts which show significant modulation at 4 hours post-treatment are genes which show a direct response to VEGF whereas at 24 hours the transcript profile may include genes which reflect survival or homeostatic functions in addition to those genes which reflect the direct effects of VEGF-A.

In addition to the different temporal profiles of transcripts, the heterogeneity of individuals was found to be very significant. Thus a number of genes which in one individual may appear to be up or down regulated in response to VEGF were found not to be consistently regulated in others. By excluding such variation, it has been possible to provide a panel of genes which are believed to be of use, particularly in conjunction with one another, in examining the true response to VEGF in human subjects.

Furthermore, the different profile of VEGF-induced expression found in serum-starved cells and non-serum-starved cells indicates the different responses that cells in the human body undergo in response to VEGF depending upon their location and nature. For example, cells in the female reproductive tract or cells undergoing radiotherapy or other treatment of a solid tumour will have a profile of response to VEGF similar to serum starved cells, whereas cells in other locations of the body are likely to respond in a manner more similar to those of the non-serum-starved cells.

In many clinical situations angiogenesis is a significant marker of clinical outcome, either desirable or undesirable. Conditions in which apoptosis is a marked or even essential feature of pathogenesis include solid tumours such as gliomas, rheumatoid arthritis, psoriasis, diabetes mellitus, SLE, stroke, Alzheimer's, dementia, hypertension, endometriosis, abnormal uterine bleeding, ovarian hyperstimiulation syndrome, pneumonia, retinopathy, macular degeneration, infertility, ovulation, peripheral vascular disease, peripheral neuropathy, atheroscelosis, vasculitis, glomerular nephritis, septicaemia, septic shock, pre-eclampsia and intrauterine growth retardation.

There is thus a continuing need for the development of reliable and robust methods for the diagnosis and prognosis of human medical conditions involving conditions associated with VEGF-A, particularly angiogenesis and vasculogenesis, including those mentioned above and elsewhere herein.

There is also a continuing need in the art to identify new targets for therapeutic intervention in such diseases. Additionally, there is a need to identify therapeutic agents with activity against such targets. Further, the use of such agents against these targets may have value in the treatment and diagnosis of these diseases.

In a first aspect, the present invention provides a method of monitoring the progression of a disease condition associated with angiogenesis or vasculogenesis in a human subject, said method comprising:
making a quantitative determination of the transcript level of at least one gene shown in table 1 in a sample of cells obtained from the site of said disease; and
comparing the transcript level so determined with the transcript level of said at least one gene obtained from a control sample of cells.

Preferably, the sample of cells are endothelial cells.

In another aspect, the invention provides a gene chip array suitable for use in the above-described method of the invention comprising at least one nucleic acid suitable for detection of at least one gene shown in Table 1; optionally a control specific for said at least one gene; and optionally at least one control for said gene chip.

In a further aspect, the invention provides assay methods for modulators of angiogenesis or vasculogenesis, wherein said method comprises:
(a) providing a protein encoded by a gene selected from Table 1;
(b) bringing said protein into contact with a candidate modulator of its activity; and
(c) determining whether said candidate modulator is capable of modulating the activity of said protein;
   or wherein said method comprises:
(a) providing an endothelial cell in culture;
(b) bringing said cell into contact with a candidate modulator of angiogenesis; and
(c) determining whether said candidate modulator is capable of modulating the transcript level of at least one gene selected from the genes of Table 1.

Modulators obtained by such methods may be used in a method of modulating angiogenesis or vasculogenesis in a human patient.

In another aspect, the identification of ESTs has allowed new potential targets for therapeutic intervention to be developed. Thus the invention provides a vector comprising an EST sequence from Table 1 operably linked to a promoter for transcription of said sequence. Such vectors are useful for expression of proteins encoded by the ESTs in the analysis of the genes in angiogenesis or vasculogenesis, and may have direct therapeutic use in themselves, e.g. as recombinant proteins or in gene therapy applications.

In another aspect, the invention provides a method of monitoring the response of a patient to treatment of a condition associated with angiogenesis or vasculogenesis which method comprises providing a sample of tissue from said patient, contacting said sample in vitro with VEGF, and determining the expression of one or more of the transcripts of Table 1. Preferably, the expression is compared to the expression of the transcripts in the sample prior to treatment with VEGF. In one aspect, the expression of one or more transcripts of Tables 1a, 1b or 1f is examined. In this aspect of the invention, where the transcripts whose expression is changed most are found to be those of Tables 1a or 1b, this will indicate that the cells have been in a state similar to serum starvation. This may be indicative of a disease state or, for example, in the case of the treatment of a tumour, an indication of a response to an anti-angiogenic therapeutic treatment. Where the expression of transcripts of Table 1f are found to have changed most, this may be indicative of cells which are not stressed and thus indicative of non-responsiveness to treatment in the case of a tumour or of healthy tissue as the case may be.

### DESCRIPTION OF THE DRAWINGS

FIG. 1a-d shows apoptosis in and cell number of cells which were treated with VEGF-A following serum withdrawal.

FIGS. 2a & b shows gene transcript levels in cells at 4 and 24 hours.

FIG. 3 shows changes in transcript levels of 3 genes.

FIG. 4 shows SAGE identifies abundant transcripts also identified on a gene chip.

### TABLES

Table 1a lists transcripts whose levels are regulated in endothelial cells treated with VEGF-A at 4 hours after treatment.

Table 1b lists transcripts whose levels are regulated in endothelial cells treated with VEGF-A at 24 hours after treatment.

Table 1c lists EST transcripts whose levels are regulated in endothelial cells at 48 hours after serum withdrawal treatment.

Table 1d lists previously characterised transcripts whose levels are regulated in endothelial cells at 48 hours after serum withdrawal treatment.

Table 1e lists further transcripts whose levels are regulated in endothelial cells at 48 hours after serum withdrawal treatment.

Table 1f lists shows transcripts whose levels are regulated by VEGF in cells which are cultured in medium supplemented with serum.

Table 2 lists transcripts abundant in endothelial cells.

Table 3 lists transcripts expressed at higher levels in HUVEC endothelial cells than in either endometrial tissue or the B lymphocyte cell line Raji.

### DETAILED DESCRIPTION OF THE INVENTION

Table 1

Reference herein to Table 1 is to be construed as meaning any one of Tables 1a, 1b, 1c, 1d, 1e and 1f, unless the context is explicitly to only one (or two or three, as the case may be) of these component parts of table 1.

Methods of Monitoring Disease Progression.

In the present invention, it will be understood that the determination of cells “obtained from the site” of disease in a patient is reference to an in vitro method practiced on a sample after removal from the body. The removal of the body sample, e.g. in a biopsy, is not part of the invention as such.

As explained above, the unique methodology used to identify the genes of Table 1 is a useful means for monitoring the progression of disease conditions associated with angiogenesis or vasculogenesis. The data we have obtained shows that some genes appear to be up-regulated in response to VEGF-A whereas others are up-regulated in conditions which lead to apoptosis of endothelial cells. Thus in treatment of diseases associated with unwanted angiogenesis, the clinician will look for a response in which the former category of genes show reduced transcript level, whereas the latter show increased transcript level.

The up or down-regulation of the genes we have identified can be made during a course of treatment of a patient so that the effectiveness of the treatment can be gauged. For example, many cancer treatments rely upon a cocktail of different anti-cancer agents. The effectiveness of any one particular cocktail may differ from patient to patient, or during the course of treatment in the patient where cells become resistant to one or more of the drugs.

In this aspect of the invention, the comparison can be made with the transcript levels obtained from the disease site of the patient at an earlier point in time, e.g. prior to treatment or between courses of treatment. Alternatively, the comparison may be made with transcript levels of cells in non-diseased tissue in said patient. Another option is to provide a control baseline sample or historical record from another patient, or, more preferably, a population of patients. Preferably, the control cells are endothelial cells.

In a preferred aspect, the invention is performed by looking at the transcript pattern of a plurality of genes. This is because we have found that in individual subjects, the transcript level of individual genes may vary. For example, in Table 1a it will be observed that in subjects 2 to 5, the cyclin D1 transcript level rose about 1.5 to 2 fold, whereas there was almost no increase in subject 1. It is therefore desirable that the transcript level is assessed for several genes. For example, the genes assessed could include at least one transcription regulator; at least one apoptosis regulator, at least one growth factor or growth factor receptor, and at least one adhesion/matrix protein.

Generally, the transcript level of at least 5, preferably at least 10 and more preferably at least 20 genes is determined.

It is also preferred that one or more of the transcript levels of table 1a or other component part of table 1 are determined.

The transcript level of a gene or genes may be determined by any suitable means. Where many different gene transcripts are being examined, a convenient method is by hybridization of the sample (either directly or after generation of cRNA or cDNA) to a gene chip array.

Where gene chip technology is used, the genes (this term used herein includes the ESTs of Table 1 are all present in commercially available chips from Affymetrix, and these chips may be used in accordance with protocols from the manufacturer. Generally, methods for the provision of microarrays and their use may also be found in, for example, WO84/01031, WO88/1058, WO89/01157, WO9.3/8472, WO95/18376/WO95/18377, WO95/24649 and EP-A-0373203 and reference may also be made to this and other literature in the art.

Table 1 provides the names of genes and these may be used to obtain their DNA sequences from databases such as Genbank. In addition, the particular sequences used on the Affymetrix chip we have used may be determined by the Affymetrix reference number supplied in the table, which are publicly available and may be related directly to Genbank reference numbers. The EST gene sequences are also given by Genbank reference numbers. Those of skill in the art may refer to either of the Affymetrix reference number of the Genbank reference number in practicing the present invention.

Alternatively, or in addition, quantitative PCR methods may be used, e.g. based upon the ABI TaqMan™ technology, which is widely used in the art. It is described in a number of prior art publications, for example reference may be made to WO00/05409. PCR methods require a primer pair which target opposite strands of the target gene at a suitable distance apart (typically 50 to 300 bases). Suitable target sequences for the primers may be determined by reference to Genbank sequences as mentioned above.

A particular application of the invention is in relation to the treatment and prognosis of diseases associated with unwanted cellular proliferation, particularly solid tumours, including gliomas and sarcomas. Such conditions rely on angiogenesis for their progression, and thus treatments which block angiogenesis or prevent the maintenance of the blood vessels are desirable.

In additions, some disease conditions associated with a lack of vasculature, such as cardiovascular disease or other conditions referred to herein above. The present invention allows such conditions to be monitored and the effectiveness of treatment regimes to be reviewed.

Gene Chips.

Although the prior art provides a gene chip which includes, as part of a very large array, the genes of one or more of Table 1a, 1b, 1c, 1d, 1e and 1f, the identification of a relatively small set of genes of diagnostic and prognostic use in the present situation allow the provision of a small chip specifically designed to be suitable use in the present invention.

Thus the invention provides a gene chip array comprising at least one nucleic acid suitable for detection of at least one gene shown in Table 1; optionally a control specific for said at least one gene; and optionally at least one control for said gene chip. Desirably, the number of sequences in the array will be such that where the number of nucleic acids suitable for detection of the Table 1 transcripts is n, the number of control nucleic acids specific for individual transcripts is n′, where n′ is from 0 to 2n, and the number of control nucleic acids (e.g. for detection of “housekeeping” transcripts, abundant endothelial cell transcripts (such as those of Table 2), transcripts which have a higher level of expression in endothelial cells (such as those of Table 3) or the like) on said gene chip is m where m is from 0 to 100, preferably from 1 to 30, then n+n′+m represent at least 50%, preferably 75% and more preferably at least 90% of the nucleic acids on said chip.

Assay Methods.

The assay method of the present invention may be practiced in a wide variety of formats, for example on protein or nucleic acid components or in whole cells in culture.

One assay comprises:
(a) providing a protein encoded by a transcript of Table 1;
(b) bringing said protein into contact with a candidate modulator of its activity; and
(c) determining whether said candidate modulator is capable of modulating the activity of said protein.

In this assay method, the determination of modulation of activity will depend upon the nature of the protein being assayed. For example, proteins with enzymatic function may be assayed in the presence of a substrate for the enzyme, such that the presence of a modulator capable of modulating the activity results in a faster or slower turnover of substrate. The substrate may be the natural substrate for the enzyme or a synthetic analogue. In either case, the substrate may be labelled with a detectable label to monitor its conversion into a final product.

For proteins with a ligand binding function, such as receptors, the candidate modulator may be examined for ligand binding function in a manner that leads to antagonism or agonism of the ligand binding property.

For proteins with DNA binding activity, such transcription regulators, the DNA binding or transcriptional activating activity may be determined, wherein a modulator is able to either enhance or reduce such activity. For example, DNA binding may be determined in a mobility shift assay.

Alternatively, the DNA region to which the protein bind may be operably linked to a reporter gene (and additionally, if needed, a promoter region and/or transcription initiation region between said DNA region and reporter gene), such that transcription of the gene is determined and the modulation of this transcription, when it occurs, can be seen. Suitable reporter genes include, for example, chloramphenicol acetyl transferase or more preferably, fluorescent reporter genes such as green fluorescent protein.

Candidate modulator compounds may be natural or synthetic chemical compounds used in drug screening programmes. Extracts of plants, microbes or other organisms, which contain several characterised or uncharacterised components may also be used. Combinatorial library technology (including solid phase synthesis and parallel synthesis methodologies) provides an efficient way of testing a potentially vast number of different substances for ability to modulate an interaction. Such libraries and their use are known in the art, for all manner of natural products, small molecules and peptides, among others. Many such libraries are commercially available and sold for drug screening programmes of the type now envisaged by the present invention.

A further class of candidate modulators are antibodies or binding fragment thereof which bind a protein target.

Example antibody fragments, capable of binding an antigen or other binding partner are the Fab fragment consisting of the VL, VH, Cl and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, a bivalent fragment including two Fab fragments linked by a disulphide bridge at the hinge region.

Single chain Fv fragments are also included. An antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047. Such a technique allows the rapid production of antibodies against an antigen, and these antibodies may then be screening in accordance with the invention.

Another class of candidate molecules are peptides based upon a fragment of the protein sequence to be inhibited. In particular, fragments of the protein corresponding to portions of the protein which interact with other proteins or with DNA may be a target for small peptides which act as competitive inhibitors of protein function. Such peptides may be for example from 5 to 20 amino acids in length.

The peptides may also provide the basis for design of mimetics. Such mimetics will be based upon analysis of the peptide to determine the amino acid residues or portions of their side chains essential and important for biological activity to define a pharmacophore followed by modelling of the pharmacophore to design mimetics which retain the essential residues or portions thereof in an appropriate three-dimensional relationship. Various computer-aided techniques exist in the art in order to facilitate the design of such mimetics.

Cell based assay methods can be configured to determine expression of the gene either at the level of transcription or at the level of translation. Where transcripts are to be measured, then this may be determined using the methods of the first aspect of the invention described above, e.g. on gene chips, by multiplex PCR, or the like.

Cell based assay methods may be used to screen candidate modulators as described above. They may also be used to screen further classes of candidate modulator, including antisense oligonucleotides. Such oligonucleotides are typically from 12 to 25, e.g. about 15 to 20 nucleotides in length, and may include or consist of modified backbone structures, e.g. methylphosphonate and phosphorothioate backbones, to help stabilise the oligonucleotide. The antisense oligonucleotides may be derived from the coding region of a target gene or be from the 5′ or 3′ untranslated region. Candidate molecules may further include RNAi, i.e. short double stranded RNA molecules which are sequence specific for a gene transcript.

Modulators obtained in accordance with the present invention may be used in methods of modulating angiogenesis or vasculogenesis in a human patient. Generally the modulator will be formulated with one or more pharmaceutically acceptable carriers suitable for a chosen route of administration to a subject. For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can for example, be prepared by dissolving, dispersing, etc, a modulator and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound(s) in an amount effective to alleviate the symptoms of the subject being treated.

Routes of administration may depend upon the precise condition being treated, though since endothelial cells form the lining of the vasculature, administration into the blood stream (e.g. by i.v. injection) is one possible route.

Vectors

The identification of a number of ESTS associated with regulation of endothelial cells by VEGF provides the basis for novel vector systems useful in the aspects of the invention described above, as well as further aspects described herein below. Thus, expression vectors for the expression of proteins encoded by the ESTs form a further aspect of the invention.

Preferably, an EST of the invention in a vector is operably linked to a control sequence which is capable of providing for the expression of the coding sequence by a host cell, i.e. the vector is an expression vector.

The term “operably linked” refers to a juxtaposition wherein the components described are in a relationship permitting them to function in their intended manner. A control sequence “operably linked” to a coding sequence is ligated in such a way that expression of the coding sequence is achieved under condition compatible with the control sequences.

Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others.

The vectors may include other sequences such as promoters or enhancers to drive the expression of the inserted nucleic acid, nucleic acid sequences so that the polypeptide is produced as a fusion and/or nucleic acid encoding secretion signals so that the polypeptide produced in the host cell is secreted from the cell.

The vectors may contain one or more selectable marker genes, for example an ampicillin resistance gene in the case of a bacterial plasmid or a neomycin resistance gene for a mammalian vector.

Vectors may further include enhancer sequences, terminator fragments, polyadenylation sequences and other sequences as appropriate.

Vectors may be used in vitro, for example for the production of RNA or used to transfect or transform a host cell. The vector may also be adapted to be used in vivo, for example in methods of gene therapy. Systems for cloning and expression of a polypeptide in a variety of different host cells are well known. Vectors include gene therapy vectors, for example vectors based on adenovirus, adeno-associated virus, retrovirus (such as HIV or MLV) or alpha virus vectors.

Promoters and other expression regulation signals may be selected to be compatible with the host cell for which the expression vector is designed. For example, yeast promoters include S. cerevisiae GAL4 and ADH promoters, S. pombe nmt1 and adh promoter. Mammalian promoters include the metallothionein promoter which is can be included in response to heavy metals such as cadmium. Viral promoters such as the SV40 large T antigen promoter or adenovirus promoters may also be used. All these promoters are readily available in the art.

Vectors for production of polypeptides encoded by the ESTs of the invention of for use in gene therapy include vectors which carry a mini-gene sequence.

Vectors may be transformed into a suitable host cell as described above to provide for expression of a polypeptide of the invention. Thus, in a further aspect the invention provides a process for preparing polypeptides encoded by ESTs according to the invention which comprises cultivating a host cell transformed or transfected with an expression vector as described above under conditions to provide for expression by the vector of a coding sequence encoding the polypeptides, and recovering the expressed polypeptides. Polypeptides may also be expressed using in vitro systems, such as reticulocyte lysate.

Polypeptides or fragments thereof in substantially isolated form encoded by ESTs of the invention form a further aspect of the present invention. Fragments of the polypeptides will preferably be at least 20 amino acids in size, and preferably from 25 amino acids up to the full length of the polypeptide.

A further aspect of the invention are nucleic acid sequences which encode said polypeptides and fragments thereof. Such nucleic acid sequences may be included in vectors such as those described above.

For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

Where an EST sequence of the present invention is present in a vector, it may be linked in-frame to a translational initiation region for translation of said sequence, or alternatively it may be in an anti-sense orientation for transcription of anti-sense RNA.

The Invention is Illustrated by the Following Examples.

Abundant and Endothelial-Biased Transcripts.

To determine the most abundant endothelial transcripts, HUVEC isolated from five different individuals were cultured to passage 5 in their optimum medium. RNA extracted from these cultures was used to prepare complex cRNA probes, which were hybridised to 12,600-element Affymetrix gene array chips (U95-A). Transcript-specificsignal data from the five hybridised chips were normalised (see methods) to allow direct inter-chip comparisons, and the median abundance of each transcript in the five cultures calculated. The top 0.5% HUVEC transcripts were clustered by function and are listed in Table 2. This experiment revealed that the five primary endothelial cultures (derived from different individuals) displayed substantial transcriptome heterogeneity. Between 6% and 8% of the 12,600 transcripts differed by >1.5-fold in abundance when the transcriptomes of the five HUVEC cultures were compared with one another.

To define the transcriptome of endothelial cells and to determine how it differs from that of other cell types, we compared the transcriptome of HUVEC with that of a B-lymphocyte cell line (Raji) and that of human endometrium. To minimise the effect of the inter-isolate heterogeneity described above, the median normalised transcript abundance in several samples of each cell/tissue type was determined—HUVEC, median of five chips: Raji, median of two chips; endometrium, median of two chips (each representing pooled tissue from five patients). Transcripts showing ten-fold higher signals in HUVECs than in either endometrium or B lymphocytes were clustered by function and are listed in Table 3. In some cases, including PAI-1, PECAM-1, collagenase and TSG-14 the signals were over fifty times higher in the endothelial cells than in either the B lymphocytes or endometrium.

VEGF-A Regulates Endothelial Cell Fate and Transcript Abundance.

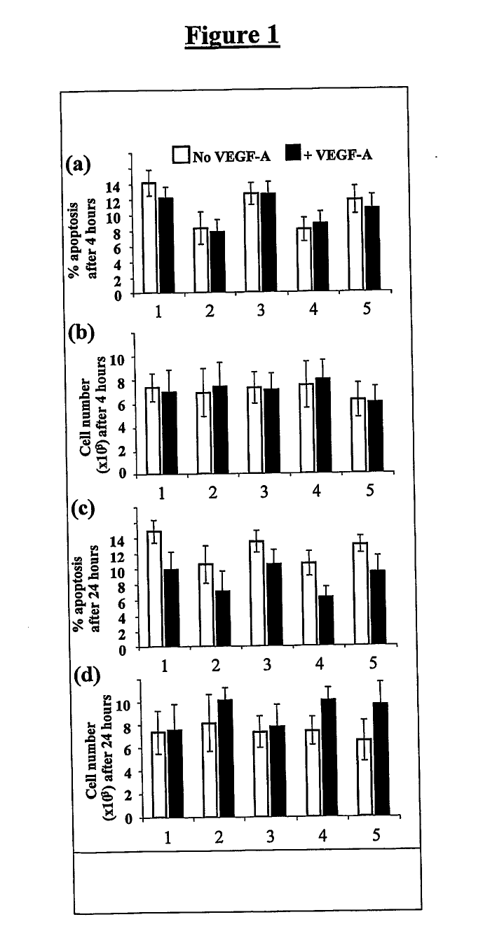

We correlated the effects of VEGF-A on endothelial cell biology and transcript abundance. In vivo, VEGF-A performs both pro-survival and mitogenic functions. To allow study of both functions in vitro, five independent primary isolates of HUVEC were cultured for 24 hr in concentrations of growth factors and serum below those required for optimal growth. This reduced the rate of proliferation and induced a low incidence of apoptosis of about 10-16%. To examine the ability of VEGF-A to reinstate proliferation and to prevent further apoptosis, the HUVEC were then cultured in the same media for a further 4 hr or 24 hr with or without 10 ng/mL VEGF-A₁₆₅. At the end of these experiments, the incidence of apoptosis and total cell number were counted and total RNA prepared. Incubation with VEGF-A for 4 hr had no significant effect on apoptosis incidence or cell number (FIG. 1 a and b). However, incubation with VEGF-A for 24 hr significantly reduced the incidence of apoptosis in all five HUVEC cultures (paired T-test P<0.05), and increased total adherent cell number in three out of the five HUVEC cultures (paired T-test P<0.05; FIG. 1 c & d).

The RNAs extracted from these cultures were used to prepare complex cRNA probes, which were hybridised to Affymetrix gene arrays as above. To determine whether VEGF-A treatment altered the overall pattern of transcript abundance in HUVEC, random effects-model analysis of variance (ANOVA) was used. This indicated that incubation with VEGF-A for 24 hr significantly altered the overall pattern of transcript abundance (F=4.8; F>3.9 implies P<0.05), but incubation with VEGF-A for 4 hr did not (F=1.3). The heterogeneity between the primary cultures noted previously was also evident in this experiment. The pattern of transcript abundance differed significantly between the five control cultures used in the 4 hr VEGF-A treatment experiment (F=7.1; F>2.4 implies P<0.05), and between the five control cultures used in the 24 hr VEGF-A treatment experiment (F=9.2; F>2.4 implies P<0.05). Interestingly, calculation of variance components based on the ANOVA showed that the change in transcript abundance pattern attributable to 24 hr of VEGF-A treatment, although significant, was only one fifth of that attributable to transcriptome differences between the five primary-cultures.

Heterogeneous Responses to VEGF-A.

ANOVA revealed that the five primary cultures differed from one another in their precise pattern of response to VEGF-A, since the statistical interaction between VEGF-A treatment and the culture source was significant (in the 24 hr experiment, F=4.4; F>2.4 implies P<0.05).

Heterogeneous responses to VEGF-A may be due to genetic and historical differences between the donors of the HUVEC, in addition to experimental errors (such as subtle variation between the precise conditions of each culture). The percentage of transcripts which, between any two cultures, differed in response to VEGF-A by >1.5-fold was determined. A duplicate vial of HUVEC from one individual (individual 3) was then thawed and cultured in an identical repeat experiment. We found that the pattern of response to VEGF-A of the two sister cultures varied less than the pattern of response to VEGF-A of unrelated cultures.

Transcripts Regulated by VEGF-A.

To identify specific transcripts regulated by either 4 hr or 24 hr incubation with VEGF-A, we selected transcripts that met three criteria; (i) Result of a Baysian T-test (CyberT algorithm; see methods) comparing abundance of the transcript in the five control and treated cultures indicated P<0.05. (ii) Abundance was regulated by VEGF-A congruently in all at least four out of the five cultures. (iii) Transcript was flagged by the Affymetrix software as being ‘present’ in the transcriptome of at least one of the cultures being compared.

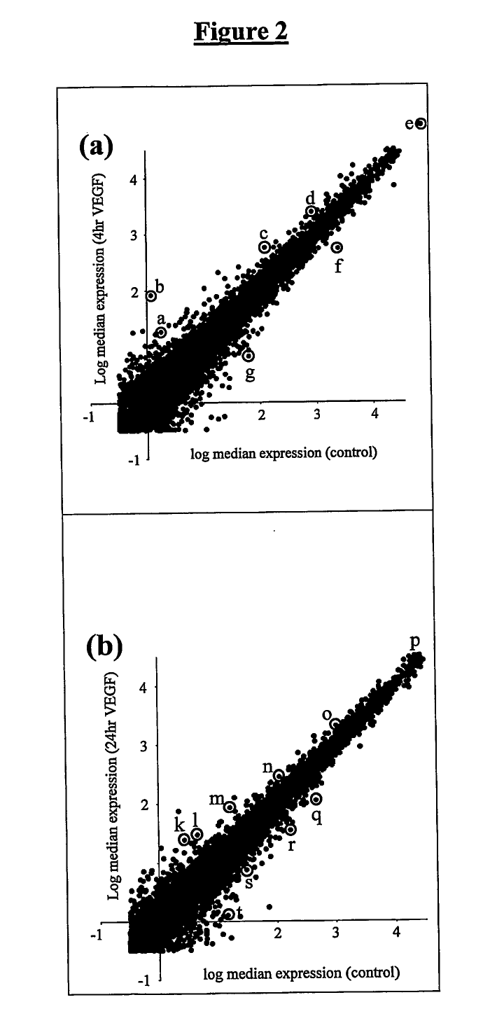

Using these criteria, we identified 20 known transcripts and 5 ESTs potentially regulated by 4 hr incubation with VEGF-A (FIG. 2a and Table 1a). We identified 55 known transcripts and 9 ESTs potentially regulated by 24 hr incubation with VEGF-A (FIG. 2b and Table 1b). Complete normalised abundance data for these transcripts is presented in Table 1a and 1b. Transcripts potentially regulated by VEGF-A encoded members of diverse protein families known to regulate endothelial cell fate, as well as uncharacterised proteins. Stromelysin-2 and the transcription factor ‘tubby’ appear likely to be regulated by VEGF-A at both the 4 hr and 24 hr time-points. Several other transcripts met the criteria listed above at either the 4 hr or 24 hr time-point, but narrowly failed the criteria at the other time point.

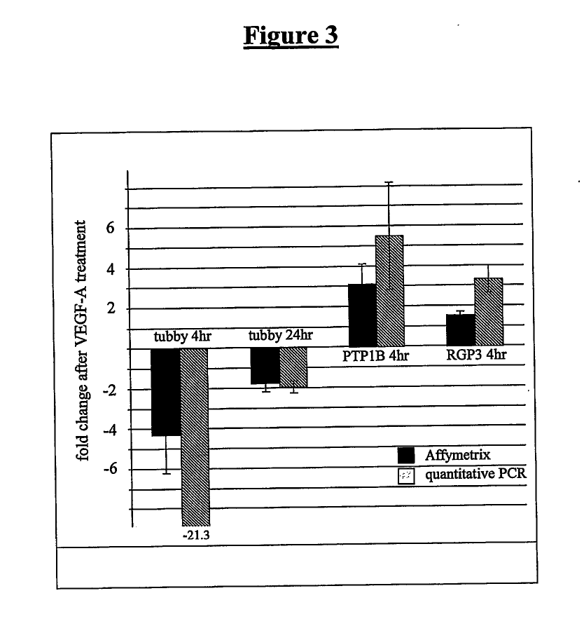

To confirm that the Affymetrix arrays had correctly identified transcripts regulated by VEGF-A, we performed quantitative real time PCR (TaqMan) using the RNAs anlaysed by Affymetrix hybridisation as templates. The Affymetrix and real-time PCR results for the three genes analysed (tubby, protein tyrosine phosphatase-1B and regulator of G-protein signalling-3) concurred. The VEGF-induced changes in transcript abundance determined by TaqMan in most cases exceeded those determined using Affymetrix array analysis (FIG. 3).

SAGE Analysis.

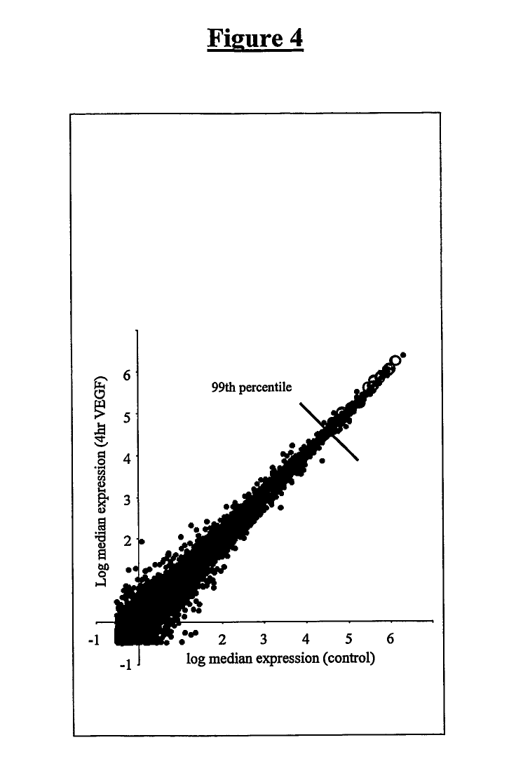

To determine the most abundant endothelial cell transcripts, and whether they were regulated by VEGF-A, we supplemented the Affymetrix gene array experiments with SAGE. A further HUVEC isolate was cultured with and without VEGF-A for 4 hr precisely as described above. Messenger RNA was isolated, and SAGE performed. A total of 5380 di-tags were sequenced from VEGF-treated cells and 6698 from untreated control cells. The list of the most abundant transcripts detected by SAGE and Affymetrix analysis largely coincided. All but five of the most abundant 0.5% of transcripts identified by SAGE were among the most abundant 1% of transcripts identified by the corresponding Affymetrix study (FIG. 4). The number of di-tags counted in this relatively small SAGE study was only sufficient to reliably assess the expression of the most abundant HUVEC transcripts. However, in agreement with the Affymetrix analysis, few if any of the most abundant HUVEC transcripts were regulated by 4 hr incubation with VEGF-A. The number of di-tags counted in the SAGE study was not sufficient to detect VEGF-mediated changes in the expression of moderate abundance transcripts, such as the changes that were detected by the more sensitive Affymetrix analysis.

Summary

Endothelial cells possess a specialised transcriptome The most abundant HUVEC transcripts included cytoskeletal elements and their regulators, ribosomal proteins, enzymes involved in carbohydrate metabolism, members of the ubiquitin system, and proteins involved in various forms of signalling (Table 2). These abundant proteins perform essential functions in diverse cell lineages and are ubiquitously expressed. Intriguingly, this list also included a non-integrin laminin receptor and a lymphokine (macrophage migration inhibitory, MIF).

Transcripts expressed more abundantly in endothelial cells than in other lineages may underlie the specialised nature of the endothelium. We expected such transcripts to be expressed at high levels in cultured endothelial cells, at moderate levels in endometrium (due to the vascular component of this tissue) and at low levels in cultured B lymphocytes. This analysis revealed that several transcripts previously known to contribute to the specialised structure and function of endothelial cells are expressed according to this pattern (Table 2). They included the serpin PAI-1 (mediates vascular healing and arterial neointima formation; [15]), matrix metalloproteinase-1 (degrades interstitial collagens during angiogenesis; [16]), and Von-Willebrand factor (which acts as a carrier for clotting factor VIIIC and mediates platelet-vessel wall interactions). Others included ERG (a member of the ETS family) and HHEX (a member of the homeobox family), which, as transcription factors, may themselves contribute to the particular nature of the endothelial transcriptome. Others transcripts expressed according to an endothelial-biased pattern encoded cell adhesion molecules such as integrins α5 & α6B, VE-cadherin [7) and CD31. These may underlie the specialised adhesion that accompanies capillary morphogenesis and transendothelial leucocyte migration. The relative abundance of growth factors to which endothelial cells specifically respond, such as VEGF-C, angiopoietin-2 and PlGF highlights the importance of their autocrine signalling and synergistic actions for endothelial cell survival [17]. Proteins encoded by the ESTs identified by this analysis may perform similarly important but as yet undefined functions in endothelial cell biology.

Responses to VEGF-A.

VEGF-A is an essential growth factor for endothelial cells, since it promotes their survival, proliferation, migration, morphogenesis into vessels, and vascular permeability. While the response of endothelial cells to VEGF-A is known to depend on post-translational signalling cascades, downstream transcriptome changes, which are currently poorly characterised may play an essential role. To define these changes, HUVEC cells were incubated with VEGF-A for both 4 hr and 24 hr. After 4 hr incubation with VEGF-A, few if any changes in proliferation and apoptosis had occurred, implying that transcript abundance changes evident at this time are direct responses to VEGF-A itself. After 24 hr incubation with VEGF-A, cell survival and proliferation had increased. Therefore, transcriptome changes at this time may reflect these processes in addition to the direct effects of VEGF-A. ANOVA indicated that 4 hr incubation with VEGF-A had a no significant effect on the global pattern of transcript abundance. Nevertheless, a small number of individual transcripts likely to be regulated by 4 hr VEGF-A incubation were identified. 24 hr exposure to VEGF-A did significantly affect the global pattern of transcript abundance. However, the change to the global transcriptome mediated by 24 hr of VEGF-A treatment was still relatively small, and less significant than the differences between the transcriptomes of endothelial cells derived from different individuals. Since this experiment was designed to investigate the acute effect of a single factor on a single cell-type, it may not be surprising to find that the abundance of only a small and select group of transcripts appears to be specifically regulated by VEGF-A. Some of these are discussed below.

VEGF-mediated control of transcripts encoding cell cycle-regulators may initiate the HUVEC proliferation shown in FIG. 1. For example, cyclin D1 (which initiates the G1/S phase transition) is up-regulated. E2F-4 (which binds to RB, p107 and p130 to suppress expression of proliferation-associated genes) is down-regulated.

The VEGF-mediated survival of HUVEC shown in FIG. 1 may be initiated by the reduced abundance of transcripts encoding pro-apoptosis proteins. The abundance of trail (a TNF-like death ligand [18]) is reduced following 4 hr VEGF-A incubation. In the HUVEC analysed in this study, the DR-5 trail receptor is very abundant (97^{th} percentile), and trail's two inhibitory decoy receptors Dcr-1 and Dcr-2 are expressed at only low levels, regardless of VEGF-A treatment. Therefore, trail may potentially act in an autocrine manner to increase the likelihood of endothelial apoptosis, and VEGF-mediated reduction in trail transcript abundance may promote endothelial survival, in addition to promoting the survival of other local cells such as vascular smooth muscle cells and leucocytes. VEGF-mediated down-regulation of transcripts encoding two other pro-apoptotic proteins may also be biologically important; p75 (enhances TNF-RI-mediated apoptosis; (19]), and DAXX (a pro-apoptosis adapter protein that associates with Fas and activates JNK pathways; [20]).

Transcript abundance changes described here may contribute to the vascular morphogenesis promoted by VEGF-A in vivo. For example, stromelysin-2, which may assist angiogenesis by degrading proteoglycans and fibronectin, is up-regulated by VEGF-A. PDGF II, which may promote arteriogenesis by acting as a vascular smooth muscle cell mitogen is also up-regulated. Up-regulation of transcripts encoding integrins β1 and α2 may also promote this process. Down-regulation of the VEGF receptor Flt-1 by VEGF-A is initially surprising. However, this may serve to limit the duration and extent of VEGF-stimulated neo-angiogenesis by negative feedback. The numerous transcription factors that appear to be regulated by VEGF-A may potentially specify VEGF-mediated changes to the transcriptome and therefore ultimately regulate the endothelial-specific proteome. Of particular interest is VEGF-mediated down-regulation of a member of the oestrogen nuclear receptor family hERR1 [21]. VEGF-A is produced by stromal cells in the endometrium in a cyclical fashion.

Therefore, down-regulation of an oestrogen receptor transcription factor by VEGF-A may allow ‘cross-talk’ between VEGF-A and reproductive steroids, to delicately control angiogenesis in reproductive tissues.

The regulation of three sets of transcripts identified here does not concord with previous studies, however there appear to be reasons for this. (i) The anti-apoptotic molecules Bcl-2 and A1 have previously been identified as VEGF-regulated [22]. However, they did not feature in our analysis since their abundance was insufficient for reliable inclusion in Affymetrix comparisons. (ii) In a previous study, continuous incubation with 50 ng/mL VEGF-A had little effect on the abundance of 588 transcripts in human microvascular endothelial cells (HMEC) [23]. However, the design of this study (investigating the long-term effects of continuous VEGF-A stimulation) and the cell type used (HMEC) may explain the disparity. (iii) VEGF-A was previously shown to up-regulate the expression of Flt-1 in HUVEC cells [13]. In our study, Flt-1 expression was not altered by 4 hr or 24 hr VEGF-A treatment but a splice variant encoding a soluble form of flt-1 was down-regulated after 24 hr. VEGF-A stimulation and Flt-1 expression may have been uncoupled in our experimental system. The Ets-1 transcription factor, which drives VEGF-mediated Flt-1 expression [16], was down-regulated by the serum withdrawal step that our HUVEC cultures underwent prior to incubation with VEGF-A (data not shown).

Although it is likely that some of the endothelial-specific and VEGF-regulated transcripts identified here will be specific to the culture system, it is equally likely that many of the transcript abundance patterns identified by this study do occur in vivo, and are functionally important in all endothelial cells. This may be confirmed by a variety of studies, such as by expressing and ‘knocking-out’ a number of the endothelial-specific and VEGF-regulated ESTs identified by this study in vascularised embryoid bodies, to assess the role they play in endothelial cells within a complex tissue.

Responses to Serum Withdrawal.

It was surprising that very few SFD-regulated transcripts were associated with a stress-induced protective response. Those that were regulated included transcripts encoding Heat Shock Protein 27 (↑2.3×), Glutathione S Transferase M4 (T9.5×) and A20 (□1.8×). Most of the transcripts traditionally associated with endothelial cell stress responses, including those up-regulated by the transcription factors NFκB, p53 and HIF-1α and heat shock factors were not up-regulated in our study—in fact, several were down-regulated. This may be due to the prolonged period of SFD chosen in our study to maximise the accumulation of apoptosis-associated transcriptional changes. This is likely to have precluded the detection of transient stress responses.

To our surprise, the overwhelming majority of SFD-dependant transcriptome changes appeared to be either directly pro-apoptotic, or to indirectly prime cells for future apoptosis. We believe that these changes may represent an essential part of the apoptotic program. Several mechanisms through which these changes are likely to support apoptosis are described below.

Transcriptome Changes Induced by Survival Factor Withdrawal are Likely to Promote Cell Death

Death receptor signaling is likely to be increased in SFD cells, since the death receptor LARD (DR3) is up-regulated ↑2× and the tumour necrosis factor homologue Trail was up-regulated ↑2.8×. Components of the apoptotic “machinery” were up-regulated in SFD cells, including Caspase 10 (↑1.8×) and Caspase 4 (↑1.7×). In SFD cells, several transcripts encoding anti-apoptotic proteins were down-regulated, including the caspase inhibitor cIAP1 (MIHB; ↓1.9×) and the DISC-associated protein TRAF-2 (↓6.1).

Down-Regulation of Survival Signals

A number of transcriptome changes appear to synergise to reduce the ability of SFD EC to respond to extra-cellular survival signals, thus promoting cell death; (i) Transcripts encoding several autocrine/paracrine EC growth and survival factors were down-regulated in the SFD cells, including VEGF-A (↓4.5×), VEGF-C (↓4.2×), Connective Tissue Growth Factor (↓1.8) and Epidermal Growth Factor (EGF; ↓5.1×). (ii) Survival factor receptors were also down-regulated. Examples included Flow-induced Endothelial G-protein-Coupled Receptor (↓4.9×), GP130 (↓5.8×) and IL1 receptor component-L1 (↓6.6×). (iii) Transcripts encoding components of the ECM, that would normally provide EC with adhesion-dependant survival signals, were also down-regulated. Examples include Collagen α2 typeVI (↓3.4×) and Collagen α1 typeVII. (↓4.3×). (iv) Adhesion molecule receptors that transduce growth/survival signals were down-regulated, including Nr-CAM (↓5.3). Interestingly, Nr-CAM is one of a small number of transcripts that are up-regulated during in vitro angiogenesis. Integrin-α2 was also significantly down-regulated (↓4.1×) however, since other integrins were up-regulated, (e.g. Integrin-α3 ↑2.9×), the significance of regulated integrin expression in SFD cells is unclear. (v) Several transcripts encoding intracellular signaling molecules that transduce survival signals in EC were down-regulated. Examples include; STAT2 (↓3.6×) and the integrin-associated kinase ICAP-1a (↓3.3×). Numerous transcripts associated with G-protein signaling were also regulated; these may be especially significant since Rho/Ras and G-protein signaling play an essential role in determining EC fate.

Transcription Factors are Regulated in Apoptotic Cultures

Transcription factors play a crucial role in controlling the apoptotic process. For example, NF-κB family members inhibit apoptosis by up-regulating expression of anti-apoptotic endothelial transcripts. Following SFD, NF-κB subunit p65 was marginally up-regulated (↑1.5×), which is not surprising given its previously described role in the response of EC to stress. However, the inhibitors of NF-κB nuclear localisation I-kBα and I-kBε (MAD3) were significantly up-regulated (2.8× and 2.7×, respectively)—this is likely to antogonise NF-κB's pro-survival effect in the SFD cells. Transcripts encoding Rel-B were also up-regulated (↑3.5×). Rel B, also known as I-Rel, is a direct inhibitor of NF-κB-mediated transcriptional activation. In addition, the NF-κB p100 subunit was up-regulated (↑4.8×). p100 has I-kB-like activity and contains a death domain. It has recently been identified as a component of a complex that sensitises cells to death receptor-mediated apoptosis and activates Caspase 8. The concept that NF-κB activity is inhibited in SFD cells is supported by the down-regulation following SFD of NF-κB-dependant transcripts such as cIAP1 and TRAF-2. The transcription factor JunD is also up-regulated by SFD (↑2.1×). By analogy with its pro-apoptotic homologue c-Jun, JunD up-regulation may promote the apoptosis of SFD EC. The abundance of a further 26 RNAs encoding transcription and splicing factors were regulated by ≧2-fold in the SFD cells—these may be responsible for some of the transcriptome changes reported here.

Transcriptional Changes May Promote Phagocytosis of Apoptotic Bodies

The final stage of the apoptotic program is engulfment of apoptotic bodies by phagocytes. Both RNA and protein of the chemokine Monocyte Chemoattractant Protein-1 (MCP-1) was undetectable in healthy EC, but they were up-regulated greatly following SFD. This de-novo MCP-1 expression may enhance the recruitment of macrophages to regions of EC death. Phagocytosis of apoptotic cells may also be promoted by the SFD-mediated up-regulation of Clusterin (↑3.7×). Clusterin (Apolipoprotein J) is induced in vital cells by apoptotic debris and phospatitidylserine-containing lipid vesicles produced when neighboring cells die, and is thought to promote the uptake of apoptotic bodies by non-professional phagocytes.

Signals Required for Mitosis are Down-Regulated by Survival Factor Deprivation

Changes in the expression of transcripts encoding regulators of the cell cycle and mitosis may underlie the mitotic arrest of serum-deprived cells, since 24 cell cycle-related transcripts were down-regulated by ≧2-fold after SFD. No cell cycle-related transcripts were up-regulated. Down-regulated transcripts included; CDC2, which is essential for G1/S and G2/M phase transitions (↓3.8×), cyclins A (↓2.9×), H (↓2.4×) and E2 (↓3.4×), proliferating cell nuclear antigen (PCNA; ↓3.4×), processivity factor for DNA polymerases (↓3.4×), and CDC45, which may play a role in loading DNA polymerase-α onto chromatin (↓3.5×).

The relevance to cell death of several other changes to transcript abundance induced during SFD were more difficult to assess. These included; Angiopoietin-2 (a promoter of vascular remodelling; ↓5.3×), Connexin 43 (a gap junction component; ↓6.0×), stromelysin II (a metalloproteinase; ↓9.1×) and Biglycan (a collagen and TGFβ-binding glycoprotein; ↑3.4×).

Based on the data presented here, we suggest that transcriptome and glycome changes may render terminally stressed cells refractory to survival signals, directly elevate death signals and caspase expression, promote cell cycle arrest, recruit phagocytes to regions of endothelial damage and promote the process of phagocytosis.

ESTs

A number of ESTs identified as relevant to the present invention are of particular interest as markers for the monitoring methods of the invention, as targets for assays, and as possible therapeutics for use in treatments. ESTs of interest have been extended and are set out in the accompanying sequence listing. Open reading frames of the ESTs may be determined and these and the ESTs or fragments thereof may be used in the present invention. Other ESTs of interest include:
AI223047 is a 1.1 kb transcript with homology to NADH dehydrogenesase(ubiquinone) 1 alpha subcomplex, with good homology to 383 bp of its sequence.
AI813532 is a 3.7 kb transcript with homology (very good homology to 1.3 kb of its length) to the A chain and R chain of the of TNF-R2, and homology to the TNF-R superfamily.
AL050021 is a 3.1 kb transcript which has homology to sco-spondin-mucin-like protein, and some homology to a potential TGF-binding protein (of M. musculus).
AB020649 is a 3.9 kb transcript with a PH domain homology, to 305 bp of its sequence and good RUN domain homology over homology to 365 bp of its sequence.
AL049701 is a 648 bp transcript with encodes a hypothetical protein, also related to clone MGC:20057.
AI885381 (710 bp) is another hypothetical protein related to clone MGC2650.
AI214965 (4.4 kb) has protein homology to the chain A, crystal structure of the C-terminal Wd40, and homology to the mRNA for KIAA1006.
AA492299 (5.6 kb) has similarity to RAP1, GTPase activating protein 1 with very good homology to 638 bp bp of its length.
AA631972 (896 bp) ishomologous to Natural Killer Transcript 4, chain A, with very good homology to 558 bp of its length.
D13633 (2.6 kb) is related to the KIAA0008 gene product.
AI720438 (925 bp) is similar to small inducible cytokine subfamily A, with protein domain homology to the solution structure of the human chemokine Hcc-2 and chain A, Nmr structure of Human Mip-1α.
M20812 (770 bp) has homology with Ig kappa chain, and protein domain homology to chain L, VEGF in complex with an affinity matured antibody and chain J, VEGF in complex with a neutralising antibody, and unigene homology to human kappa-Immunoglobulin germline pseudogene.
AI985964 (487 bp) has homology to trefoil factor 3 (intestinal), with protein domain homology to chain A.
S73591 (2.7 kb) is homolgous to a protein upregulated by 1,25-dihydroxyvitamin D-3.
AI912041 (723 bp) is similar to heat shock 10 KD protein 1, with protein domain homology to the chain A of heat shock protein 1.
U41635 (2.7 kb) is a protein amplified in osteosarcoma, and has protein domain homology to chain A of human Guanylate binding protein-1. Also unigene homology to human OS-9 precursor mRNA.
U79259 (1.7 kb) is similar to atrophin-1-human protein.
AI760932 (805 bp) has similarity to prostaglandin D2 synthase and protein domain homology to chain B, crystal structure of human neutrophil.
X66436 (1.9 kb) has homology to a human GTP-binding protein-like GTPase of uknknown function
AB014538 (50.1 kb) has homology to Chain S, cryo-Em structure of the of the heavy meromysin.
AF052106 (4.2 kb) is homologous to the hypothetical protein MGC 4614.
Y09022 (1.4 kb) has homology to a not-like protein and protein domain homology to chain A of melanin protein.
D80008 (3.3 kb) is homologous to KIAA0186.
AI743606 (1.9 KB) has homology to a ras-related protein and protein domain homology to chain A/crystal structure of sec4-guanosine-5′.
AA663800 (1.4 kb) is a hypothetical protein.
   Heterogeneity Between Primary Cultures.

A significant finding in this study was that primary endothelial cultures derived from different individuals displayed substantial transcriptome heterogeneity. A component of the heterogeneity may be attributable to genetic and historical differences between the individuals from which the cultures were derived. This was supported by the fact that duplicate cultures of the same individual's cells displayed less differences in their responses to VEGF-A than cultures derived from different individuals. It is probable that similar differences in response to VEGF-A may also occur in individual patients treated with VEGF-A based therapies for coronary artery [26] and peripheral vascular disease [27]. Since duplicate cultures of the same individual's cells still retain some transcriptome differences, other components of transcriptome heterogeneity must also exist, such as slight variations in culture conditions. We therefore suggest that it is extremely unwise to draw conclusions from genomics studies employing single, possibly idiosyncratic primary cell cultures.

Interpretation of Transcript Abundance Data.

Affymetrix expression data is now sometimes accepted without further verification by an alternative technique [28]. However, to ensure our data was robust, we have used SAGE to validate the relative abundance of a large set of highly expressed transcripts, and quantitative real-time PCR to validate the regulation of three transcripts by VEGF-A. We believe that the reliability of Affymetrix expression data is critically dependent on stringent quality control and careful global & local normalisation of the raw data, as described in the methods. Due to the large number of transcripts interrogated by the Affymetrix arrays, some ‘false positive’ transcript abundance changes congruent in all five in VEGF-treated cultures were expected by chance. This is a problem common to all large-scale genomics studies. Techniques such as Bonferroni corrections can be used to elevate the P-values required for significance according to the number of genes being observed, and techniques such as ‘Significance Analysis of Microarrays’ [29] can be used to estimate the false discovery rate. However, the most robust method to reduce ‘false positive’ transcript abundance changes is to use multiple independent samples, as we have done here.

Summary

We have identified a specialised endothelial cell-specific pattern of transcript abundance (transcriptome) that is regulated by VEGF-A. This unique transcriptome is likely to underlie the specialised structure of these cells and the unique roles they play in vivo during both health and disease. The endothelial-specific and VEGF-regulated transcripts identified by this study provide insights into the pre-translational events that lead to the complex processes regulated by VEGF (including endothelial cell survival, tissue invasion and interaction with other cell types). It also provides new targets for the treatment of angiogenesis-dependant diseases such as cancer, endometriosis and arteriosclerosis. This study also provides a warning. We have shown that the transcriptomes of primary endothelial cells isolated from different patients are surprisingly heterogeneous. This is likely to also be the case with other cell types. Therefore, we suggest that experiments conducted on single (possibly idiosyncratic) primary cell cultures may be misleading.

Materials and Methods

Cell Culture and RNA Isolation for Gene Array Studies.

HUVEC were isolated from umbilical cords by collagenase digestion as described [30]. After culture to passage 2, several vials of each HUVEC isolate were frozen for future use. After thawing, HUVEC were cultured to passage 5 in a humidified atmosphere of 5% CO₂ using proprietary culture medium (large vessel endothelial cell medium; TCS, Botolph, UK) supplemented with a proprietary mixture of heparin, hydrocortisone, EGF, FGF, 2% foetal calf serum, gentamicin and amphotericin. Once at passage 5, HUVEC were partially deprived of growth factors by culturing in the basal medium supplemented with only 2% charcoal-stripped FCS (Gibco/BRL UK) in the presence or absence of 10 ng/mL human VEGF₁₆₅ (R & D systems Abingdon UK). Identical confluence and identical batches of medium, serum and VEGF-A were used for each HUVEC culture. Total RNA was prepared using Trizol (Gibco/BRL UK) followed by passage through a RNeasy column (Qiagen, UK) and ethanol precipitation. RNA integrity and concentration was assessed using an Agilent 2100 bioanalyser.

Assessment of Apoptosis and Cell Number

The HUVEC isolates used for gene array analysis were concurrently cultured in 48-well plates using the conditions described above. Total and apoptotic adherent cells were enumerated in 8 replicate wells using an epifluorescent relief-phase contrast microscope (Olympus, UK). Apoptotic cells were defined as those which excluded trypan blue (0.2%; Sigma UK) and propidium iodide (20 μg/mL; Sigma), but which labelled with AnnexinV (Annexin V-Fluos staining kit used according to the manufacturer's instructions; Roche UK) and which also showed morphological characteristics of apoptosis.

Affymetrix Oligonucleotide Gene Arrays

Biotin-labelled cRNA complex probes were prepared and hybridised to Affymetrix Human “U95A” gene-chips according to Affymetrix protocols (Affymetrix, High Wycombe, UK). The quality of the expression data from all chips was assessed using both Affymetrix Microarray Suite (version 4.0) and dChip [31] software. Data from chips that failed these quality control tests was discarded. Transcript abundance data (‘average differences’) were globally scaled to bring the median gene expression of each chip (excluding control genes) to 1. A minor degree of local scaling was then required to ensure that the expression of transcripts of every expression level on all chips was comparable. To achieve this, the ‘loess’ function of the ‘R’ statistical software system (http://www.r-project.org/) was used, based on a method used by the ‘NOMAD’ protocol (http://pevsnerlab.kennedykrieger.org/). Normalised transcript abundance data from VEGF-treated and un-treated cultures was then compared using the CyberT algorithm (version 7.03; sliding window=301, Bayes confidence estimate=15). This algorithm is an unpaired T-test, modified by the inclusion of a Bayesian prior based on the variance of other transcripts in the data set [32]. Detailed Affymetrix probe set hybridisation data for selected genes was examined using a Filemaker Pro database system. This system allowed the formation of clusters based on both data from the Affymetrix chips (reported transcript abundance, individual probe set metrics, etc) and on known functionality. The system then allowed these clusters to be combined in multiple-comparison statements' (AND/OR/NOT) to yield smaller datasets, which in turn were linked-out to web databases (eg, Swiss Prot, BLAST, etc) for the collection of sequence and functional information. For further statistical analysis, the ‘R’ statistical software system and Microsoft Excel 2001 were used on a Macintosh G4 computer.

SAGE Procedure and Computation

A further isolate of HUVEC was purchased from TCS (Botolph Claydon, UK) and cultured as above with and without 10 ng/mL VEGF-A₁₆₅ for 4 hr. SAGE libraries were generated from 5 □g polyA+ RNA following the SAGE protocol previously described with minor modifications [33]. Captured cDNAs were ligated to linkers that contained a recognition site for the tagging enzyme BsmF1 (New England Biolabs). SAGE tags were then released with BsmF1, blunt ended, and ligated head to head to form di-tags. These were released from linkers by Nla III digestion, concatenated and cloned into de-phosphorylated Sph I cut pGEM-3Zf+ (Promega Life Sciences), sequenced using the Applied Biosystems Prism Dye Terminator reaction kit and run on an ABI 373 automated sequencer (Applied Biosystems Warrington UK).

Real Time PCR

The ABI PRISM 7700 Sequence Detection System (TaqMan) was used to perform real-time polymerase chain reactions according to the manufacturers protocols. For all RNAs used in the Affymetrix study, C_{T} values for three transcripts were compared to those for cyclophilin. Primers and probes used were;

(i) Tubby;

| | |
|---|---|
| FORWARD | |
| 5′-CCCCCCAGGGTATCACCA-3′ | (SEQ ID NO: 4) |
| REVERSE | |
| 5′-CCCCGGTCCATCCCTTT-3′ | (SEQ ID NO: 5) |
| probe FAM- | |
| 5′-AAATGCCGCATCACTCGGGACAAT-3′-TAMRA | (SEQ ID NO: 6) |

(ii) PTP-LB;

| | |
|---|---|
| FORWARD | |
| 5′-TGATCCAGACAGCCGACCA-3′ | (SEQ ID NO: 7) |
| REVERSE | |
| 5′-CCCATGATGAATTTGGCACC-3′ | (SEQ ID NO: 8) |
| probe FAM- | |
| 5′-AAATGCCGCATCACTCGGGACAAT-3′- | (SEQ ID NO: 9) |
| TAMRA. | |

(iii) RGS-3

| | |
|---|---|
| FORWARD | |
| 5′-GGCTGCTTCGACCTGGC-3′ | (SEQ ID NO: 10) |
| REVERSE | |
| 5′-AAGCGAGGGTACGAGTCCTTT-3′ | (SEQ ID NO: 11) |
| probe FAM- | |
| 5′-AGAAGCGCATCTTCGGGCTCATGGT-3′- | (SEQ ID NO: 12) |
| TAMRA | |

Detailed Figure & Table Legends

Table 1a& b. Candidate VEGF-regulated transcripts that pass the statistical tests described in the text are listed in functional clusters. The direction of abundance change is denoted in some cases. By-P denotes the P-value from a Bayesian T-test used to compare transcript abundance in the five pairs of control and VEGF-treated cultures. Probe set denotes the Affymetrix code corresponding to each transcript. Cyclophilin, which is overall not significantly regulated by VEGF-A is shown as a control.

Table 1a The most abundant 0.5% of HUVEC transcripts are listed. Abundance refers to median normalised transcript abundance in five HUVEC cultures from different individuals (where the transcript of median abundance has been assigned a value of to 1). Probe set denotes the Affymetrix probe set corresponding to each transcript.

Table 1b Normalised transcript abundance data for candidate VEGF-regulated HUVEC transcripts that met statistical criteria described in the text is shown (for each chip the transcript of median abundance has been assigned a value of to 1). 1-5 denote HUVEC from five individuals cultured with (VEGF) and without (con) VEGF-A. By-P denotes the P-value from a Bayesian T-test used to compare transcript abundance in five pairs of control and VEGF-treated cultures. Probe set denotes the Affymetrix code corresponding to each transcript.

Table 1c & d. Table 1c provides ESTs according to the invention whose transcript level was found to be modulated after 48 hours serum withdrawal. These ESTs are thus indicative of an apoptopic state. Table 1d indicates genes with known function also with significantly modulated transcript levels.

Table 1e. Table 1e provides additional transcripts which are found to be modulated after 48 hours serum withdrawal. These were determined as described herein for Table 1c.

Table 1f. Table 1f provides transcripts which were found to be regulated by treatment with VEGF of primary HUVECs isolated from umbilical cords of three individuals by collagenase digestion and cultured to passage 5 in a fully humidified atmosphere of 5% CO₂ in basal culture medium supplemented with a proprietary mixture of heparin, hydrocortisone, epidermal growth factor, fibroblast growth factor, 2% foetal calf serum (FCS), gentamycin and amphotericin (large vessel endothelial cell medium; TCS, Botolph, UK). Cells' were treated with 10 ng/ml VEGF 165 for 24 hours. Data from the three samples were analysed and the average fold-change expression is shown in the final column of the table.

Table 2. Abundant transcripts as described above.

Table 3. Transcripts that were at least ten-fold more abundant in HUVEC than in both B-lymphocytes and endometrium are listed. Et/BL denotes ratio of normalised transcript abundance in HUVEC (median of 5 chips) to normalised abundance in the human B-lymphocyte line Raji (median of 2 chips). Et/Em denotes ratio of normalised abundance in HUVEC to normalised abundance in samples of human endometrium (median of 2 chips, each representing pooled tissue from five individuals).

FIG. 1. VEGF-A inhibits apoptosis and induces proliferation of primary endothelial cells. (a and b) HUVEC were cultured with (black bars) or without (clear bars) VEGF-A for 4 hrs. (c and d) HUVEC were cultured with or without VEGF-A for 24 hrs. (a and c) Mean incidence of apoptosis. (b and d) Mean cell number. Results for 5 separate endothelial cell isolates are shown, error bars denote two SD.

FIG. 2. VEGF-regulated transcripts. Dot-plots were used to compare logₑ (normalised transcript abundance) in HUVEC cultured with (Y-axis) or without (X-axis) 10 ng/mL VEGF-A. (a) 4 hrs VEGF-A. (b) 24 hr VEGF-A. Lower case letters refer to transcripts listed in Table 3. Note that the most abundant transcripts are not shown, in order to expand the lower section of the scale.

FIG. 3. Quantitative PCR confirmed a set of results from the Affymetrix gene array analysis. The fold-difference between transcript abundance in control and VEGF-treated HUVEC is shown. Figures represent median abundance in five cultures, and are relative to the abundance of cyclophilin (probe set 33667_at; not regulated substantially by VEGF-A). The same RNAs were used for PCR and Affymetrix analysis. Error bars denote the standard errors of the mean. Transcripts analysed were tubby (34600_s_at; abundance assessed after both 4 hr and 24 hr treatment with VEGF-A), protein tyrosine phosphatase-1B (40137_at; 4 hrs VEGF-A) and regulator of G-protein signalling-3 (36737_at; 4 hrs VEGF-A).

FIG. 4. SAGE identifies the same abundant endothelial cell transcripts as Affymetrix analysis. A dot-plot is shown of logₑ (normalised transcript abundance) in HUVEC cultured with (Y-axis) or without (X-axis) 10 ng/mL VEGF-A for 4 hrs. Overlaid white circles show the position in the Affymetrix datasets of the most abundant 0.5% of transcripts detected by SAGE. A line marks the 99^{th} percentile of the Affymetrix data.

Abbreviations

Serial Analysis of Gene Expression; SAGE
vascular endothelial growth factor; VEGF
mitogen activated protein kinase; MAPK
stress-activated protein kinase; SAPK
c-jun-NH2-kinase; JNK
focal adhesion kinase; FAK
human umbilical vein endothelial cell(s); HUVEC
analysis of variance; ANOVA
human microvascular endothelial cells; HMEC

### REFERENCES

1. Ferrara N, Carver-Moore K, Chen H, Dowd M, Lu L, O'Shea K S, Powell-Braxton L, Hillan K J, Moore M W: Heterozygous embryonic lethality induced by targeted inactivation of the VEGF gene. Nature 1996; 380:439-442.
2. Carmeliet P, Ferreira V, Breier G, Pollefeyt S, Kieckens L, Gertsenstein M, Fahrig M, Vandenhoeck A, Harpal K, Eberhardt C, Declercq C, Pawling J, Moons L, Collen D, Risau W, Nagy A: Abnormal blood vessel development and lethality in embryos lacking a single VEGF allele. Nature 1996; 380:435-439.
3. Benjamin L E, Golijanin D, Itin A, Pode D, Keshet E: Selective ablation of immature blood vessels in established human tumors follows vascular endothelial growth factor withdrawal [see comments]. J Clin Invest 1999; 103:159-165.
4. Alon T, Hemo I, Itin A, Pe'er J, Stone J, Keshet E: Vascular endothelial growth factor acts as a survival factor for newly formed retinal vessels and has implications for retinopathy of prematurity. Nat Med 1995; 1:1024-1028.
5. Gale N W, Yancopoulos G D: Growth factors acting via endothelial cell-specific receptor tyrosine kinases: VEGFs, angiopoietins, and ephrins in vascular development. Genes Dev 1999; 13:1055-1066.
6. Gerber H P, McMurtrey A, Kowalski J, Yan M, Keyt B A, Dixit V, Ferrara N: Vascular endothelial growth factor regulates endothelial cell survival through the phosphatidylinositol 3′-kinase/Akt signal transduction pathway. Requirement for Flk-1/KDR activation. J Biol Chem 1998; 273:30336-30343.
7. Carmeliet P, Lampugnani M G, Moons L, Breviario F, Compernolle V, Bono F, Balconi G, Spagnuolo R, Oostuyse B, Dewerchin M, Zanetti A, Angellilo A, Mattot V, Nuyens D, Lutgens E, Clotman F, de Ruiter M C, Gittenberger-de Groot A, Poelmann R, Lupu F, Herbert J M, Collen D, Dejana E: Targeted deficiency or cytosolic truncation of the VE-cadherin gene in mice impairs VEGF-mediated endothelial survival and angiogenesis. Cell 1999; 98:147-157.
8. Ilan N, Mahooti S, Madri J A: Distinct signal transduction pathways are utilized during the tube formation and survival phases of in vitro angiogenesis. J Cell Sci 1998; 111:3621-3631.
9. Gupta K, Kshirsagar S, Li W, Gui L, Ramakrishnan S, Gupta P, Law P Y, Hebbel R P: VEGF prevents apoptosis of human microvascular endothelial cells via opposing effects on MAPK/ERK and SAPK/JNK signaling. Exp Cell Res 1999; 247:495-504.
10. Abu-Ghazaleh R, Kabir J, Jia H; Lobo M, Zachary I: Src mediates stimulation by vascular endothelial growth factor of the phosphorylation of focal adhesion kinase at tyrosine 861, and migration and anti-apoptosis in endothelial cells. Biochem J 2001; 360:255-264.
11. Zeng H, Zhao D, Mukhopadhyay D: Flt-1-mediated down-regulation of endothelial cell proliferation through pertussis toxin-sensitive G proteins, bg subunits, small GTPase CDC42- and partly by Rac-1. J Biol Chem 2001; 28:28.
12. Bussolati B, Dunk C, Grohman M, Kontos C D, Mason J, Ahmed A: Vascular endothelial growth factor receptor-1 modulates vascular endothelial growth factor-mediated angiogenesis via nitric oxide. Am J Pathol 2001; 159:993-1008.
13. Sato Y, Kanno S, Oda N, Abe M, Ito M, Shitara K, Shibuya M: Properties of two VEGF receptors, Flt-1 and KDR, in signal transduction. Ann N Y Acad Sci 2000; 902:201-205; discussion 205-207.
14. Clark D E, Smith S K, He Y, Day K A, Licence D R, Corps A N, Lammoglia R, Charnock-Jones D S: A vascular endothelial growth factor antagonist is produced by the human placenta and released into the maternal circulation. Biol Reprod 1998; 59:1540-1548.
15. Carmeliet P, Moons L, Lijnen R, Janssens S, Lupu F, Collen D, Gerard R D: Inhibitory role of plasminogen activator inhibitor-1 in arterial wound healing and neointima formation: a gene targeting and gene transfer study in mice. Circulation 1997; 96:3180-3191.
16. Iwasaka C, Tanaka K, Abe M, Sato Y: Ets-1 regulates angiogenesis by inducing the expression of urokinase-type plasminogen activator and matrix metalloproteinase-1 and the migration of vascular endothelial cells. J Cell Physiol 1996; 169:522-531.
17. Carmeliet P, Moons L, Luttun A, Vincenti V, Compernolle V, De Mol M, Wu Y, Bono F, Devy L, Beck H, Scholz D, Acker T, DiPalma T, Dewerchin M, Noel A, Stalmans I, Barra A, Blacher S, Vandendriessche T, Ponten A, Eriksson U, Plate K H, Foidart J M, Schaper W, Charnock-Jones D S, Hicklin D J, Herbert J M, Collen D, Persico M G: Synergism between vascular endothelial growth factor and placental growth factor contributes to angiogenesis and plasma extravasation in pathological conditions. Nat Med 2001; 7:575-583.
18. Wiley S R, Schooley K, Smolak P J, Din W S, Huang C P, Nicholl J K, Sutherland G R, Smith T D, Rauch C, Smith C A, et al.: Identification and characterization of a new member of the TNF family that induces apoptosis. Immunity 1995; 3:673-682.
19. Gupta S: Molecular Steps of Tumor Necrosis Factor Receptor-Mediated Apoptosis. Current Molecular Medicine 2001; 1:317-324.
20. Yang X, Khosravi-Far R, Chang H Y, Baltimore D: Daxx, a novel Fas-binding protein that activates JNK and apoptosis. Cell 1997; 89:1067-1076.
21. Yang N, Shigeta H, Shi H, Teng C T: Estrogen-related receptor, hERR1, modulates estrogen receptor-mediated response of human lactoferrin gene promoter. J Biol Chem 1996; 271:5795-5804.
22. Gerber H P, Dixit V, Ferrara N: Vascular endothelial growth factor induces expression of the antiapoptotic proteins Bcl-2 and A1 in vascular endothelial cells. J Biol Chem 1998; 273:13313-13316.
23. Vasile E, Tomita Y, Brown L F, Kocher O, Dvorak H F: Differential expression of thymosin {beta}-10 by early passage and senescent vascular endothelium is modulated by VPF/VEGF: evidence for senescent endothelial cells in vivo at sites of atherosclerosis. Faseb J 2001; 15:458-466.
24. Gerritsen M E: Functional heterogeneity of vascular endothelial cells. Biochem Pharmacol 1987; 36:2701-2711.
25. Craig L E, Spelman J P, Strandberg J D, Zink M C: Endothelial cells from diverse tissues exhibit differences in growth and morphology. Microvasc Res 1998; 55:65-76.
26. Losordo D W, Vale P R, Symes J F, Dunnington C H, Esakof D D, Maysky M, Ashare A B, Lathi K, Isner J M: Gene therapy for myocardial angiogenesis: initial clinical results with direct myocardial injection of phVEGF165 as sole therapy for myocardial ischemia. Circulation 1998; 98:2800-2804.
27. Simovic D, Isner J M, Ropper A H, Pieczek A, Weinberg D H: Improvement in chronic ischemic neuropathy after intramuscular phVEGF165 gene transfer in patients with critical limb ischemia. Arch Neurol 2001; 58:761-768.
28. Akalin E, Hendrix R C, Polavarapu R G, Pearson T C, Neylan J F, Larsen C P, Lakkis F G: Gene expression analysis in human renal allograft biopsy samples using high-density oligoarray technology. Transplantation 2001; 72:948-953.
29. Tusher V G, Tibshirani R, Chu G: Significance analysis of microarrays applied to the ionizing radiation response. Proc Natl Acad Sci USA 2001; 98:5116-5121.
30. Li X F, Charnock-Jones D S, Zhang E, Hiby S, Malik S, Day K, Licence D, Bowen J M, Gardner L, King A, Loke Y W, Smith S K: Angiogenic growth factor messenger ribonucleic acids in uterine natural killer cells. J Clin Endocrinol Metab 2001; 86:1823-1834.
31. Li C, Wong W H: Model-based analysis of oligonucleotide arrays: expression index computation and outlier detection. Proc Natl Acad Sci USA 2001; 98:31-36.
32. Long A D, Mangalam H J, Chan B Y, Tolleri L, Hatfield G W, Baldi P: Improved statistical inference from DNA microarray data using analysis of variance and a Bayesian statistical framework. Analysis of global gene expression in Escherichia coli K12. J Biol Chem 2001; 276:19937-19944.

33. Velculescu V E, Zhang L, Vogelstein B, Kinzler K W: Serial analysis of gene expression. Science 1995; 270:484-487.

TABLE 1a
Transcripts regulated by 4 hr VEGF-A

| | | | | | | | | | | | | |
|---|---|---|---|---|---|---|---|---|---|---|---|---|
| | 1 | 1 | 2 | 2 | 3 | 3 | 4 | 4 | 5 | 5 | | |
| Transcript | CON | VEGF | CON | VEGF | CON | VEGF | CON | VEGF | CON | VEGF | By-P | Probe set |

Transcription regulators

| | | | | | | | | | | | | |
|---|---|---|---|---|---|---|---|---|---|---|---|---|
| NPW38 | 3.35 | 1.88 | 3.97 | 3.13 | 3.13 | 1.99 | 8.10 | 3.43 | 4.18 | 0.47 | 0.0078 | 34325_at |
| CDC25 B | 3.39 | 1.75 | 4.64 | 3.11 | 5.37 | 3.31 | 3.92 | 3.71 | 2.74 | 0.56 | 0.0488 | 1347_at |
| cyclin D1 | 25.74 | 26.59 | 68.59 | 93.88 | 40.58 | 65.21 | 39.56 | 67.10 | 32.86 | 79.94 | 0.0134 | 38418_at |
| HEM45 | 1.70 | 4.01 | 2.50 | 3.85 | 1.32 | 3.81 | 2.94 | 3.66 | 2.06 | 3.08 | 0.0195 | 33304_at |
| tubby transcription factor | 5.48 | 4.93 | 5.17 | 0.52 | 5.33 | 2.68 | 4.93 | 4.53 | 4.36 | 1.34 | 0.0112 | 34600_s_at |

Apoptosis regulators

| | | | | | | | | | | | | |
|---|---|---|---|---|---|---|---|---|---|---|---|---|
| TRAIL | 2.92 | 1.07 | 2.54 | 1.04 | 1.47 | 1.11 | 0.75 | 0.57 | 3.24 | 1.25 | 0.0153 | 1715_at |
| TNF receptor II (p75) | 6.57 | 2.07 | 3.81 | 2.56 | 4.13 | 2.52 | 5.26 | 3.29 | 5.65 | 4.57 | 0.0153 | 33813_at |

Growth factors/receptors

| | | | | | | | | | | | | |
|---|---|---|---|---|---|---|---|---|---|---|---|---|
| PDGF 2 (c-sis) | 9.40 | 20.32 | 12.79 | 12.52 | 13.73 | 26.01 | 13.24 | 17.75 | 11.77 | 21.79 | 0.0087 | 1573_at |
| IGF-BP10 | 21.87 | 29.54 | 25.70 | 40.11 | 25.08 | 40.09 | 21.38 | 31.94 | 25.29 | 40.13 | 0.0198 | 38772_at |
| neuropilin-2 | 4.15 | 1.52 | 4.44 | 0.61 | 2.49 | 3.09 | 1.47 | 1.08 | 3.39 | 2.50 | 0.0307 | 33853_s_at |

Adhesion/Matrix

| | | | | | | | | | | | | |
|---|---|---|---|---|---|---|---|---|---|---|---|---|
| stromelysin-2 | 1.16 | 1.39 | 0.52 | 2.80 | 0.87 | 2.25 | 1.51 | 2.94 | 1.13 | 1.81 | 0.0132 | 1006_at |

Miscellaneous

| | | | | | | | | | | | | |
|---|---|---|---|---|---|---|---|---|---|---|---|---|
| cytokeratin 17 | 0.44 | 4.68 | 1.36 | 4.59 | 6.45 | 6.83 | 1.08 | 8.63 | 0.53 | 9.26 | 0.0002 | 34301_r_at |
| Pex14 | 2.16 | 0.55 | 1.05 | 0.70 | 2.59 | 0.51 | 3.38 | 1.15 | 1.90 | 0.94 | 0.0012 | 33760_at |
| Na, K-ATPase beta-1 | 5.10 | 8.00 | 7.47 | 17.01 | 10.04 | 12.90 | 8.21 | 16.33 | 12.16 | 15.81 | 0.0121 | 37669_s_at |
| Hsp70-5 | 32.95 | 46.72 | 26.81 | 35.84 | 38.04 | 44.61 | 30.12 | 58.40 | 35.75 | 62.17 | 0.0207 | 36614_at |
| calponin 3 | 9.89 | 9.17 | 8.54 | 12.13 | 9.33 | 13.33 | 10.29 | 17.53 | 8.51 | 16.49 | 0.0308 | 40953_at |
| PTP 1B | 0.45 | 2.47 | 1.63 | 1.74 | 0.51 | 1.28 | 1.90 | 2.30 | 1.26 | 3.47 | 0.0344 | 40137_at |
| Regulator of G-protein sig. 3 | 14.12 | 23.80 | 13.23 | 16.45 | 18.94 | 24.25 | 19.61 | 22.89 | 19.70 | 37.30 | 0.0366 | 37637_at |
| cyclophilin (control) | 182.23 | 169.68 | 178.60 | 184.98 | 182.39 | 172.08 | 170.81 | 186.10 | 172.13 | 143.69 | 0.5526 | 33667_at |

ESTs

| | | | | | | | | | | | | |
|---|---|---|---|---|---|---|---|---|---|---|---|---|
| EST AA883101 | 3.65 | 0.52 | 0.52 | 0.86 | 3.90 | 0.52 | 5.52 | 2.79 | 4.21 | 0.77 | 0.0009 | 39815_at |
| EST D80007 | 0.52 | 2.68 | 0.52 | 2.72 | 0.52 | 1.51 | 0.52 | 0.55 | 0.60 | 1.37 | 0.0020 | 34731_at |
| EST AF000959 | 38.29 | 39.37 | 73.24 | 40.65 | 37.82 | 26.30 | 48.65 | 34.26 | 53.96 | 22.59 | 0.0121 | 38995_at |
| EST AL050021 | 4.85 | 7.30 | 6.70 | 10.18 | 7.70 | 7.81 | 8.08 | 11.75 | 4.72 | 14.57 | 0.0174 | 39748_at |
| EST AF052172 | 2.43 | 2.95 | 1.32 | 2.39 | 1.02 | 3.00 | 1.41 | 2.43 | 1.13 | 2.40 | 0.0273 | 36747_at |

TABLE 1b
Transcripts regulated by 24 hr VEGF-A

| | | | | | | | | | | | | |
|---|---|---|---|---|---|---|---|---|---|---|---|---|
| | 1 | 1 | 2 | 2 | 3 | 3 | 4 | 4 | 5 | 5 | | |
| Transcript | CON | VEGF | CON | VEGF | CON | VEGF | CON | VEGF | CON | VEGF | By-P | Probe set |

Transcription regulators

| | | | | | | | | | | | | |
|---|---|---|---|---|---|---|---|---|---|---|---|---|
| hERR1 | 6.15 | 3.20 | 6.36 | 3.90 | 5.05 | 0.79 | 6.07 | 3.57 | 8.53 | 5.10 | 0.006 | 1487_at |
| Proto-Oncogene C-Myc | 13.50 | 12.03 | 9.84 | 8.37 | 11.33 | 8.17 | 8.81 | 4.93 | 20.34 | 7.02 | 0.0333 | 1936_s_at |
| PBX1 | 3.46 | 2.67 | 2.01 | 1.29 | 2.14 | 1.60 | 2.68 | 0.54 | 9.47 | 2.58 | 0.0264 | 32063_at |
| LMO2 | 4.30 | 7.52 | 5.89 | 7.67 | 5.38 | 6.23 | 5.78 | 9.00 | 5.07 | 7.24 | 0.0375 | 32184_at |
| fra-1 | 3.29 | 1.96 | 2.97 | 2.43 | 3.23 | 2.15 | 3.90 | 2.90 | 6.77 | 2.75 | 0.0476 | 32271_at |
| Tubby | 6.75 | 4.32 | 3.54 | 2.26 | 4.00 | 1.56 | 3.63 | 1.79 | 4.77 | 3.84 | 0.0442 | 34600_s_at |
| neuronal PAS1 | 2.59 | 0.88 | 2.33 | 0.52 | 1.17 | 0.52 | 2.05 | 2.13 | 5.02 | 0.52 | 0.0055 | 34652_at |
| TFIIF | 15.34 | 10.38 | 9.85 | 9.51 | 11.91 | 9.25 | 10.74 | 8.41 | 24.03 | 9.61 | 0.0378 | 36826_at |
| SCML2 | 1.39 | 2.39 | 1.06 | 2.86 | 1.50 | 2.38 | 2.64 | 3.35 | 0.39 | 1.84 | 0.0136 | 38518_at |
| E2F-4 | 19.37 | 13.50 | 11.50 | 11.89 | 14.61 | 10.04 | 10.49 | 8.01 | 30.41 | 11.01 | 0.0284 | 38707_r_at |
| DRAP1 | 7.69 | 12.88 | 11.35 | 14.18 | 10.93 | 17.10 | 4.59 | 4.70 | 9.31 | 15.55 | 0.0454 | 39077_at |
| R kappa B | 8.23 | 6.06 | 8.03 | 4.56 | 7.88 | 4.59 | 7.05 | 4.84 | 9.93 | 4.90 | 0.0174 | 39137_at |
| HOX3D | 4.33 | 3.44 | 6.35 | 1.57 | 4.46 | 0.73 | 5.57 | 3.93 | 7.17 | 3.84 | 0.004 | 416_s_at |

DNA repair

| | | | | | | | | | | | | |
|---|---|---|---|---|---|---|---|---|---|---|---|---|
| OGG1 | 5.85 | 2.88 | 4.62 | 2.22 | 5.57 | 1.45 | 2.68 | 1.98 | 12.01 | 3.92 | 0.0043 | 34146_at |

Apoptosis regulators

| | | | | | | | | | | | | |
|---|---|---|---|---|---|---|---|---|---|---|---|---|
| DAXX | 8.46 | 5.76 | 6.38 | 4.15 | 7.30 | 4.65 | 8.56 | 5.87 | 12.71 | 4.93 | 0.0155 | 1754_at |

Growth factors

| | | | | | | | | | | | | |
|---|---|---|---|---|---|---|---|---|---|---|---|---|
| activin beta-C | 2.74 | 2.75 | 3.64 | 1.89 | 3.52 | 0.54 | 2.69 | 1.50 | 8.97 | 2.39 | 0.0103 | 35915_at |
| growth/differentiation | 5.85 | 3.40 | 2.74 | 2.61 | 3.44 | 1.26 | 3.31 | 2.51 | 20.00 | 3.63 | 0.0266 | 887_at |
| factor 1 | | | | | | | | | | | | |

Adhesion/Matrix

| | | | | | | | | | | | | |
|---|---|---|---|---|---|---|---|---|---|---|---|---|
| stromelysin-2 | 1.35 | 3.42 | 0.98 | 1.96 | 4.42 | 5.21 | 3.17 | 4.24 | 1.62 | 5.13 | 0.0231 | 1006_at |
| collagen C-proteinase enh. | 3.39 | 1.11 | 2.57 | 2.32 | 1.72 | 0.52 | 2.77 | 1.25 | 14.18 | 1.73 | 0.0132 | 31609_s_at |
| integrin beta 1 | 60.23 | 73.00 | 75.84 | 91.95 | 67.92 | 80.28 | 62.31 | 85.33 | 57.31 | 91.49 | 0.0027 | 32808_at |
| procollagen C-proteinase | 5.61 | 2.63 | 5.11 | 4.06 | 7.31 | 3.76 | 8.40 | 6.44 | 17.91 | 3.77 | 0.0097 | 39406_at |
| integrin alpha-2 | 1.13 | 3.00 | 4.02 | 5.35 | 4.78 | 7.98 | 1.68 | 2.69 | 0.39 | 2.64 | 0.0329 | 41481_at |

Cell-surface receptors

| | | | | | | | | | | | | |
|---|---|---|---|---|---|---|---|---|---|---|---|---|
| interleukin-8 receptor type B | 3.79 | 2.14 | 2.38 | 1.61 | 3.04 | 1.63 | 2.43 | 1.56 | 6.29 | 2.95 | 0.0242 | 1032_at |
| Flt-1 | 2.31 | 1.42 | 3.25 | 1.86 | 2.24 | 0.53 | 2.71 | 0.89 | 4.73 | 2.49 | 0.0091 | 1567_at |
| IGF-binding protein-3 | 6.05 | 2.39 | 1.51 | 2.20 | 3.74 | 0.79 | 2.43 | 0.66 | 20.70 | 2.19 | 0.0116 | 1586_at |
| LDL receptor related | 8.82 | 6.08 | 5.80 | 5.64 | 13.48 | 3.19 | 6.16 | 4.45 | 34.61 | 4.36 | 0.0061 | 31815_r_at |
| protein 3 | | | | | | | | | | | | |
| prostaglandin E receptor | 7.39 | 4.58 | 5.54 | 4.10 | 3.55 | 2.86 | 5.08 | 3.76 | 20.75 | 5.06 | 0.0314 | 32691_s_at |
| EP3 | | | | | | | | | | | | |
| dopamine D4 receptor | 1.25 | 0.81 | 2.57 | 0.52 | 3.37 | 0.52 | 3.68 | 0.52 | 10.04 | 2.95 | 0.0039 | 35042_at |
| glutamate receptor type 4 | 21.16 | 16.08 | 15.46 | 13.10 | 19.03 | 14.34 | 13.94 | 11.84 | 44.46 | 13.91 | 0.0199 | 35485_at |
| Leukosialin | 2.67 | 2.09 | 2.29 | 1.98 | 2.51 | 0.53 | 2.67 | 1.73 | 3.87 | 0.55 | 0.0137 | 36798_g_at |
| erythropoietin receptor | 18.68 | 14.69 | 11.17 | 8.82 | 12.85 | 6.08 | 11.47 | 9.17 | 68.03 | 10.15 | 0.0158 | 396_f_at |
| leukotriene b4 receptor | 2.58 | 5.09 | 2.51 | 4.60 | 2.34 | 2.51 | 2.69 | 3.44 | 0.45 | 4.74 | 0.0036 | 39624_at |
| DMBT1 | 6.29 | 4.31 | 2.61 | 2.89 | 4.67 | 1.06 | 4.01 | 2.41 | 9.83 | 3.04 | 0.0135 | 41382_at |

Miscellaneous

| | | | | | | | | | | | | |
|---|---|---|---|---|---|---|---|---|---|---|---|---|
| c-Ral | 16.26 | 23.51 | 20.62 | 22.34 | 27.48 | 34.40 | 22.52 | 30.25 | 18.86 | 32.63 | 0.0344 | 1877_g_at |
| RAP1 | 6.06 | 4.67 | 7.40 | 6.10 | 5.20 | 4.84 | 6.03 | 3.84 | 41.79 | 5.97 | 0.0365 | 33080_s_at |
| cytidine deaminase | 7.71 | 5.31 | 5.71 | 2.03 | 7.48 | 0.48 | 4.94 | 3.93 | 10.12 | 4.61 | 0.0037 | 1117_at |
| cytochrome P450 IIA | 3.00 | 2.08 | 1.72 | 0.52 | 1.88 | 0.79 | 2.00 | 1.06 | 2.49 | 1.53 | 0.0345 | 1553_r_at |
| Calreticulin | 65.41 | 41.33 | 39.49 | 37.99 | 45.98 | 18.25 | 50.46 | 40.82 | 62.61 | 40.92 | 0.0061 | 32543_at |
| ribosomal S6 kinase | 4.62 | 7.03 | 2.68 | 3.84 | 5.28 | 5.33 | 0.97 | 2.43 | 2.58 | 7.35 | 0.0334 | 32892_at |
| HGF activator inhibitor | 6.30 | 0.69 | 2.49 | 0.51 | 1.65 | 2.83 | 3.12 | 1.38 | 16.66 | 2.82 | 0.009 | 33448_at |
| ADP-ribosylation factor- | 2.00 | 4.24 | 2.21 | 2.58 | 1.88 | 3.79 | 2.12 | 3.01 | 0.42 | 2.70 | 0.0063 | 33796_at |
| like 4 | | | | | | | | | | | | |
| BAF170 | 4.32 | 1.08 | 1.48 | 1.18 | 1.79 | 0.52 | 3.43 | 1.97 | 3.60 | 0.68 | 0.0031 | 34690_at |
| cytochrome c oxidase VIIb | 5.13 | 7.58 | 5.41 | 7.08 | 5.62 | 9.59 | 5.28 | 7.22 | 4.54 | 7.22 | 0.0179 | 36687_at |
| GlcNAc alpha-sialyltrans. | 3.35 | 5.25 | 0.64 | 2.55 | 2.14 | 4.23 | 1.53 | 2.86 | 0.39 | 1.99 | 0.006 | 36916_at |
| FEZ1-T | 7.80 | 4.34 | 6.20 | 4.01 | 7.05 | 4.42 | 5.32 | 2.68 | 56.47 | 5.73 | 0.021 | 37744_r_at |
| membrane cofactor protein | 8.83 | 12.09 | 9.43 | 10.43 | 10.64 | 15.54 | 8.02 | 13.90 | 11.80 | 15.93 | 0.037 | 38441_s_at |
| lysosomal acid lipase | 2.56 | 3.35 | 1.01 | 1.80 | 3.11 | 4.80 | 1.90 | 2.88 | 0.40 | 2.12 | 0.0483 | 38745_at |
| thymus specific peptidase | 3.51 | 1.79 | 2.08 | 1.10 | 2.70 | 2.33 | 2.36 | 1.99 | 13.60 | 1.67 | 0.0243 | 39306_at |
| cullin-1 | 2.80 | 3.69 | 2.10 | 4.20 | 3.52 | 4.79 | 3.42 | 3.99 | 1.24 | 3.17 | 0.0451 | 39724_s_at |
| Fzr1 | 9.15 | 6.04 | 4.11 | 3.20 | 4.34 | 2.29 | 5.29 | 3.02 | 13.43 | 4.60 | 0.0226 | 39855_at |
| MAP kinase phosphatase 4 | 3.85 | 0.61 | 4.02 | 1.43 | 3.19 | 0.71 | 3.95 | 2.35 | 6.80 | 0.55 | 0.0002 | 40186_at |
| phosphodiesterase I alpha | 6.33 | 2.87 | 0.47 | 0.72 | 2.11 | 1.17 | 3.01 | 0.57 | 14.30 | 4.71 | 0.0331 | 41125_r_at |
| 5-nucleotidase | 2.40 | 2.77 | 1.90 | 3.02 | 1.78 | 3.75 | 2.40 | 3.23 | 1.90 | 3.25 | 0.0343 | 738_at |
| cyclophilin (control) | 86.10 | 73.36 | 83.25 | 90.25 | 76.10 | 73.76 | 93.12 | 80.47 | 77.19 | 61.09 | 0.2545 | 35823_at |

ESTs

| | | | | | | | | | | | | |
|---|---|---|---|---|---|---|---|---|---|---|---|---|
| EST AB014574 | 15.22 | 14.04 | 18.63 | 11.71 | 14.77 | 9.60 | 17.09 | 14.29 | 22.85 | 13.53 | 0.039 | 31826_at |
| EST AL050065 | 2.91 | 1.44 | 2.58 | 2.03 | 3.13 | 1.12 | 2.25 | 1.17 | 2.85 | 1.48 | 0.0177 | 34112_r_at |
| EST AA527880 | 3.96 | 6.17 | 4.86 | 5.76 | 3.34 | 4.27 | 3.94 | 4.01 | 1.49 | 5.53 | 0.0438 | 35773_i_at |
| EST AB020649 | 0.99 | 2.95 | 1.27 | 3.94 | 1.82 | 4.28 | 2.99 | 4.05 | 0.39 | 4.29 | 0.0001 | 36150_at |
| EST AI140857 | 4.48 | 3.01 | 2.38 | 2.31 | 2.87 | 1.10 | 3.06 | 1.46 | 31.98 | 2.69 | 0.0291 | 37429_g_at |
| EST W28610 | 9.72 | 3.25 | 7.77 | 6.28 | 9.90 | 5.51 | 7.20 | 5.11 | 11.09 | 4.71 | 0.0054 | 38942_r_at |
| EST AB028951 | 1.47 | 2.71 | 2.11 | 3.18 | 2.78 | 4.45 | 2.68 | 3.38 | 0.48 | 2.00 | 0.0348 | 39417_at |
| EST AB011148 | 1.97 | 2.64 | 2.96 | 3.44 | 2.89 | 4.16 | 1.44 | 3.36 | 1.30 | 2.82 | 0.0406 | 40811_at |
| EST W26628 | 14.77 | 9.77 | 10.09 | 6.33 | 14.71 | 6.54 | 14.14 | 8.81 | 14.81 | 9.33 | 0.006 | 41514_s_at |

TABLE 1c
Transcripts potentially regulated by 48 hour serum withdrawal treatment

| | | | Direction of |
|---|---|---|---|
| | | | regulation |
| Accession number | Bays P | Probe set | by S/W |
| AI214965 | 1.2767E−09 | 34130_at | DOWN |
| AA492299 | 2.0876E−09 | 33081_at | DOWN |
| AI985964 | 1.5923E−08 | 37897_s_at | DOWN |
| N42007 | 1.7283E−08 | 40564_at | DOWN |
| AA255502 | 2.203E−08 | 39969_at | DOWN |
| AI912041 | 2.2559E−07 | 39353_at | DOWN |
| AI267373 | 2.7539E−07 | 34273_at | DOWN |
| AI680675 | 6.2944E−07 | 41569_at | DOWN |
| AA704137 | 1.6396E−06 | 39395_at | UP |
| W73046 | 2.2218E−06 | 35467_g_at | DOWN |
| AA128249 | 2.2615E−06 | 38430_at | DOWN |
| AA522537 | 2.8653E−06 | 39367_at | DOWN |
| W07033 | 7.0103E−06 | 35261_at | DOWN |
| H68340 | 9.3845E−06 | 41446_f_at | DOWN |
| AI130910 | 1.1059E−05 | 37050_r_at | DOWN |
| AI126004 | 1.2425E−05 | 33150_at | DOWN |
| AI740522 | 1.812E−05 | 38085_at | DOWN |
| W52024 | 1.9632E−05 | 34317_g_at | UP |
| W63793 | 2.1867E−05 | 36685_at | DOWN |
| AI688098 | 2.5998E−05 | 33458_r_at | DOWN |
| AA418437 | 2.8084E−05 | 34246_at | UP |
| AA845349 | 3.9339E−05 | 37348_s_at | DOWN |
| AI201108 | 8.4431E−05 | 38338_at | UP |
| AI701164 | 9.31E−05 | 37662_at | DOWN |
| AI928365 | 9.6083E−05 | 38267_at | DOWN |
| AA195301 | 0.0001098 | 34805_at | DOWN |
| AI885381 | 0.00014249 | 36529_at | UP |
| R93527 | 0.00018273 | 39594_f_at | DOWN |
| AI400011 | 0.00018588 | 40257_at | UP |
| AL079283 | 0.00019364 | 34813_at | DOWN |
| AA151716 | 0.00019721 | 32720_at | DOWN |
| AI765533 | 0.0002168 | 34335_at | DOWN |
| AI539439 | 0.00022743 | 35726_at | DOWN |
| N50520 | 0.00025606 | 36687_at | DOWN |
| AI674208 | 0.00026549 | 40239_g_at | UP |
| AI806379 | 0.00029566 | 39844_at | DOWN |
| AA194159 | 0.00034278 | 41282_s_at | UP |
| AA883502 | 0.00035449 | 40505_at | UP |
| AA932443 | 0.00039397 | 41624_r_at | UP |
| W52024 | 0.00043052 | 34316_at | UP |
| H16917 | 0.00046224 | 39879_s_at | UP |
| AA746355 | 0.00048502 | 37244_at | DOWN |
| AA873266 | 0.00050258 | 36720_at | DOWN |
| W68046 | 0.00061729 | 35154_at | UP |
| AI200373 | 0.00071031 | 34157_f_at | DOWN |
| H12458 | 0.00072262 | 2090_i_at | UP |
| AI246726 | 0.00079684 | 37046_at | DOWN |
| AI677689 | 0.00087305 | 40223_r_at | UP |
| AA487755 | 0.00089282 | 38761_s_at | UP |
| AL079292 | 0.00089292 | 39140_at | DOWN |
| AA768912 | 0.00101371 | 39086_g_at | DOWN |
| AI222594 | 0.00107765 | 41229_at | UP |
| AA058852 | 0.00129805 | 40986_s_at | UP |
| R92331 | 0.00140036 | 36130_f_at | DOWN |
| AI971726 | 0.00154908 | 34508_r_at | UP |
| AA905543 | 0.00159635 | 38620_at | DOWN |
| AI039880 | 0.00177015 | 37358_at | DOWN |
| AI803447 | 0.00190313 | 37337_at | DOWN |
| AI961743 | 0.00199838 | 38823_s_at | DOWN |
| T75292 | 0.00203617 | 33173_g_at | DOWN |
| AI201243 | 0.0020658 | 35963_at | DOWN |
| AA917945 | 0.00223194 | 35991_at | DOWN |
| AI075181 | 0.00227451 | 35882_at | DOWN |
| AL109689 | 0.00236391 | 34673_r_at | DOWN |
| AI800499 | 0.00243417 | 32112_s_at | UP |
| AA827795 | 0.00273835 | 41340_at | UP |
| AA426364 | 0.00279902 | 38751_i_at | UP |
| AI347088 | 0.00292432 | 35738_at | DOWN |
| AI827793 | 0.00298312 | 39516_at | DOWN |
| AI935551 | 0.00317125 | 35734_at | DOWN |
| AI377866 | 0.00332516 | 39870_at | DOWN |
| AA663800 | 0.00332676 | 39910_at | UP |
| AA059408 | 0.00337212 | 38676_at | DOWN |
| AA127624 | 0.00345583 | 33865_at | DOWN |
| W02490 | 0.00356828 | 40038_at | UP |
| AI052224 | 0.00363555 | 33016_at | UP |
| AA152202 | 0.00386074 | 32222_at | DOWN |
| AW007731 | 0.00389989 | 39092_at | DOWN |
| AI925946 | 0.00393206 | 35067_at | DOWN |
| AA203213 | 0.00400675 | 38432_at | UP |
| R87876 | 0.00417282 | 39798_at | UP |
| H97470 | 0.00525231 | 39518_at | DOWN |
| AA156987 | 0.00559231 | 39162_at | DOWN |
| AA149428 | 0.00574025 | 32789_at | DOWN |
| AL109701 | 0.0058121 | 36948_at | DOWN |
| AL109682 | 0.00595769 | 34538_at | UP |
| AI827895 | 0.0062797 | 36224_g_at | UP |
| AA926957 | 0.00691371 | 40982_at | DOWN |
| AI057115 | 0.00741561 | 40601_at | DOWN |
| AI720438 | 0.00745117 | 33790_at | DOWN |
| AA478904 | 0.00771475 | 34216_at | DOWN |
| AI095508 | 0.00793104 | 33207_at | DOWN |
| AA152406 | 0.00817228 | 39031_at | UP |
| AI127424 | 0.00856099 | 38251_at | UP |
| AA203476 | 0.00886895 | 40412_at | DOWN |
| AA877795 | 0.00923382 | 33854_at | DOWN |
| AA426364 | 0.0093988 | 38752_r_at | UP |
| R93981 | 0.00996441 | 41331_at | DOWN |

TABLE 1d

| | | | Direction of |
|---|---|---|---|
| Accession | | | regulation by |
| number | Probe set | identity | S/W |
| X07820 | 1006_at | metalloproteinase stromelysin-2 | DOWN |
| M12886 | 1105_s_at | T-cell receptor active beta-chain mRNA | DOWN |
| U43916 | 1321_s_at | tumor-associated membrane protein homolog (TMP) | DOWN |
| M31166 | 1491_at | tumor necrosis factor-inducible (TSG-14) | DOWN |
| M12783 | 1573_at | c-sis/platelet-derived growth factor 2 (SIS/PDGF2) | UP |
| X56681 | 1612_s_at | Human junD | UP |
| U65410 | 1721_g_at | Mad2 (hsMAD2) | DOWN |
| U08023 | 1786_at | cellular proto-oncogene (c-mer) mRNA | UP |
| J05614 | 1824_s_at | proliferating cell nuclear antigen (PCNA) | DOWN |
| U01134 | 1964_g_at | soluble vascular endothelial cell growth factor recep | DOWN |
| X17033 | 1978_at | integrin alpha-2 subunit | DOWN |
| M14752 | 2041_i_at | Human c-abl gene | DOWN |
| U12255 | 31432_g_at | Human IgG Fc receptor hFcRn mRNA | UP |
| S73591 | 31508_at | brain-expressed HHCPA78 homolog | UP |
| K01383 | 31623_f_at | Human metallothionein-I-A gene | DOWN |
| U81554 | 31670_s_at | Homo sapiens CaM kinase II isoform mRNA | DOWN |
| Z98744 | 31751_f_at | histone H4 | DOWN |
| U34802 | 31778_at | Human intrinsic membrane protein MP70 (Cx50) gene | DOWN |
| Y13492 | 31830_s_at | Homo sapiens mRNA for smoothelin | DOWN |
| D87735 | 31907_at | Homo sapiens mRNA for ribosomal protein L14 | UP |
| U56421 | 31921_at | Human olfactory receptor (OLF3) gene | DOWN |
| L02870 | 32123_at | Human alpha-1 type VII collagen (COL7A1) | DOWN |
| X17042 | 32227_at | hematopoetic proteoglycan core protein | DOWN |
| X56841 | 32321_at | H. sapiens HLA-E | UP |
| D87012 | 32362_r_at | Human (lambda) DNA for immunoglobin light chain | DOWN |
| X55954 | 32395_r_at | Human mRNA for HL23 ribosomal protein homologue | UP |
| X52947 | 32531_at | Human mRNA for cardiac gap junction protein | DOWN |
| AJ131186 | 33230_at | nuclear matrix protein NMP200 | DOWN |
| U86782 | 33247_at | 26S proteasome-associated pad1 homolog (POH1) | DOWN |
| S66213 | 33410_at | integrin alpha 6B | DOWN |
| AF058921 | 33707_at | Homo sapiens cytosolic phospholipase A2-gamma | UP |
| AF056085 | 33764_at | Homo sapiens GABA-B receptor mRNA | DOWN |
| M36200 | 33780_at | Human synaptobrevin 1 (SYB1) | DOWN |
| X13794 | 33820_g_at | H. sapiens lactate dehydrogenase B gene exon 1 and 2 | DOWN |
| J05243 | 33833_at | Human nonerythroid alpha-spectrin (SPTAN1) | UP |
| AB008109 | 33890_at | Homo sapiens mRNA for RGS5 | DOWN |
| AJ001019 | 34075_at | Homo sapiens mRNA for RNF3A (DONG1) | DOWN |
| Z26876 | 34085_at | H. sapiens gene for ribosomal protein L38 | UP |
| U27768 | 34272_at | Human RGP4 | DOWN |
| M28225 | 34375_at | Human JE gene encoding a monocyte secretory protein | UP |
| V00511 | 34552_at | Human mRNA encoding pregastrin | DOWN |
| M12963 | 34638_r_at | class I alcohol dehydrogenase (ADH1) alpha | DOWN |
| X83535 | 34747_at | membrane-type matrix metalloproteinase | DOWN |
| U41766 | 34761_r_at | MDC9 | DOWN |
| AB019987 | 34878_at | chromosome-associated polypeptide-C | DOWN |
| AB012130 | 34936_at | SBC2 mRNA for sodium bicarbonate cotransporter2 | DOWN |
| U94333 | 35036_at | Human Clq/MBL/SPA receptor C1qR(p) | DOWN |
| D63391 | 35800_at | platelet activating factor acetylhydrolase IB gamma | UP |
| D00265 | 35818_at | Homo sapiens mRNA for cytochrome c | DOWN |
| D42123 | 35828_at | Homo sapiens mRNA for ESP1/CRP2 | UP |
| L19161 | 35934_at | translation initiation factor eIF-2 gamma subunit | DOWN |
| M72393 | 35938_at | calcium-dependent phospholipid-binding protein | DOWN |
| AF067656 | 35995_at | Homo sapiens ZW10 interactor Zwint | DOWN |
| M72709 | 36098_at | Human alternative splicing factor mRNA | DOWN |
| X16277 | 36203_at | Human gene for ornithine decarboxylase ODC | DOWN |
| Z12173 | 36262_at | GNS mRNA encoding glucosamine-6-sulphatase | DOWN |
| U72649 | 36634_at | Human BTG2 (BTG2) | UP |
| X78947 | 36638_at | H. sapiens mRNA for connective tissue growth factor | DOWN |
| M29065 | 36654_s_at | Human hnRNP A2 protein | DOWN |
| AF072099 | 36753_at | immunoglobulin-like transcript 3 protein variant 1 | DOWN |
| M25915 | 36780_at | Human complement cytolysis inhibitor (CLI) | UP |
| Z23090 | 36785_at | H. sapiens mRNA for 28 kDa heat shock protein | UP |
| M19267 | 36791_g_at | Human tropomyosin mRNA | UP |
| Z24727 | 36792_at | H. sapiens tropomyosin isoform mRNA | UP |
| AF016050 | 36836_at | VEGF165 | DOWN |
| U75679 | 36913_at | Human histone stem-loop binding protein (SLBP) | DOWN |
| X59618 | 36922_at | small subunit ribonucleotide reductase | DOWN |
| U16954 | 36941_at | Human (AF1q) mRNA | DOWN |
| U41635 | 36996_at | Human OS-9 precurosor mRNA | UP |
| X82209 | 37283_at | H. sapiens MN1 | UP |
| X04828 | 37307_at | Human mRNA for G(i) protein alpha-subunit | UP |
| X01060 | 37324_at | Human mRNA for transferrin receptor | DOWN |
| X63692 | 37333_at | DNA (cytosin-5)-methyltransferase | DOWN |
| X58536 | 37383_f_at | Human mRNA for HLA class I locus C heavy chain | UP |
| U97188 | 37558_at | Homo sapiens putative RNA binding protein KOC (koc) | DOWN |
| U27655 | 37637_at | Human RGP3 mRNA | UP |
| M69039 | 37668_at | Human pre-mRNA splicing factor SF2p32 | DOWN |
| U16799 | 37669_s_at | Human Na,K-ATPase beta-1 subunit mRNA | UP |
| M22382 | 37720_at | mitochondrial matrix protein P1 (nuclear encoded) | DOWN |
| Y07909 | 37762_at | H. sapiens mRNA for Progression Associated Protein | DOWN |
| X12654 | 37927_at | Human mRNA for cell cycle gene RCC1 | DOWN |
| X55110 | 38124_at | Human mRNA for neurite outgrowth-promoting protein | UP |
| J04599 | 38126_at | biglycan | UP |
| AL049650 | 38455_at | (small nuclear ribonucleoprotein particle) protein B | DOWN |
| AF054183 | 38708_at | Homo sapiens GTP binding protein mRNA | DOWN |
| X64229 | 38992_at | H. sapiens dek | DOWN |
| AB024704 | 39109_at | Homo sapiens mRNA for fls353 | DOWN |
| M37583 | 39337_at | Human histone (H2A.Z) | DOWN |
| M31516 | 39695_at | Human decay-accelerating factor mRNA | DOWN |
| AF000364 | 39792_at | heterogeneous nuclear ribonucleoprotein R mRNA | DOWN |
| M94856 | 39799_at | fatty acid binding protein homologue (PA-FABP) | DOWN |
| M98343 | 39861_at | Homo sapiens amplaxin (EMS1) mRNA | UP |
| AB000449 | 39980_at | Homo sapiens mRNA for VRK1 | DOWN |
| D84557 | 40117_at | Homo sapiens mRNA for HsMcm6 | DOWN |
| X14850 | 40195_at | Human H2A.X mRNA encoding histone H2A.X | DOWN |
| D12763 | 40322_at | Homo sapiens mRNA for ST2 | DOWN |
| X61498 | 40362_at | H. sapiens mRNA for NF-kB | UP |
| U41387 | 40490_at | Human Gu protein mRNA | DOWN |
| AB008375 | 40681_at | osteoblast specific cysteine-rich protein | DOWN |
| X54942 | 40690_at | H. sapiens ckshs2 mRNA for Cks1 protein homologue | DOWN |
| L41498 | 40886_at | Homo sapiens longation factor 1-alpha 1 (PTI-1) mRNA | DOWN |
| U46751 | 40898_at | Human phosphotyrosine independent ligand p62 | UP |
| D29805 | 40960_at | Human mRNA for beta-1,4-galactosyltransferase | UP |
| AF043101 | 41072_at | Homo sapiens caveolin-3 | DOWN |
| U32519 | 41133_at | Human GAP SH3 binding protein mRNA | DOWN |
| AF029750 | 41168_at | Homo sapiens tapasin (NGS-17) | UP |
| X74039 | 41169_at | urokinase plasminogen activator receptor | DOWN |
| AB013382 | 41193_at | Homo sapiens mRNA for DUSP6 | DOWN |
| D32129 | 41237_at | Human mRNA for HLA class-I (HLA-A26) heavy chain | UP |
| X17033 | 41481_at | Human mRNA for integrin alpha-2 | DOWN |
| X56681 | 41483_s_at | Human junD | UP |
| L15189 | 41510_s_at | Homo sapiens mitochondrial HSP75 mRNA | DOWN |
| U95735 | 41517_g_at | Human SNARE protein Ykt6 (YKT6) mRNA | DOWN |
| M62424 | 41700_at | Human thrombin receptor mRNA | DOWN |
| AF061034 | 41742_s_at | Homo sapiens FIP2 | UP |
| AF061034 | 41743_i_at | Homo sapiens FIP2 | UP |
| U63717 | 467_at | osteoclast stimulating factor mRNA | DOWN |
| U57452 | 481_at | SNF1-like protein kinase mRNA | DOWN |
| M94250 | 577_at | retinoic acid inducible factor (MK) | UP |
| L78833 | 605_at | BRCA1, Rho7 and vatI genes, complete cds | UP |
| M10321 | 607_s_at | Human von Willebrand factor | UP |
| U90313 | 824_at | glutathione-S-transferase homolog mRNA | DOWN |
| U12471 | 867_s_at | Human thrombospondin-1 gene | DOWN |
| M26683 | 875_g_at | interferon gamma treatment inducible mRNA | UP |
| X74794 | 981_at | H. sapiens P1-Cdc21 | DOWN |

TABLE 1e

| | | | Direction of |
|---|---|---|---|
| Accession | | | regulation |
| number | Probe set | Identity | by S/W |
| AF004327 | 1951_at | angiopoietin 2 | DOWN |
| AF012023 | 40843_at | ICAP-1a | DOWN |
| AF015257 | 37447_at | Flow-induced Endothelial | DOWN |
| | | G-protein-Coupled Receptor | |
| AF050145 | 39451_i_at | iduronate-2-sulphatase | UP |
| AF091433 | 35249_at | Cyclin E2 | DOWN |
| AJ223728 | 37458_at | CDC45 | DOWN |
| D87673 | 720_at | heat shock transcription factor-4 | UP |
| HG2855- | 1179_at | Heat Shock Protein 70 | DOWN |
| HT2 | | | |
| L08069 | 39118_at | DNAJ | DOWN |
| M37197 | 32194_at | CCAAT transcription binding | DOWN |
| | | factor subunit g | |
| M38258 | 1587_at | retinoic acid receptor-gamma | UP |
| M57230 | 37621_at | GP130 | DOWN |
| M59911 | 884_at | Integrin alpha 3 | UP |
| M65188 | 2018_at | connexin 43 | DOWN |
| M69043 | 1461_at | IkB alpha | UP |
| M77810 | 1071_at | GATA-2 | UP |
| M83221 | 570_at | Rel-B (I-Rel) | UP |
| M96233 | 556_s_at | Glutathione S Transferase M4 | UP |
| U11791 | 1924_at | Cyclin H | DOWN |
| U12597 | 33784_at | TRAF2 | DOWN |
| U15590 | 528_at | heat shock protein 17/3 | DOWN |
| U18671 | 36770_at | STAT2 | DOWN |
| U18932 | 34182_at | heparan sulphate N-deacetylase | DOWN |
| | | Nsulphotransferase | |
| U28014 | 195_s_at | Caspase 4 | UP |
| U37518 | 1715_at | TRAIL | UP |
| U37547 | 36578_at | cIAP1 (MIHB) | DOWN |
| U55258 | 37288_g_at | Nr-CAM | DOWN |
| U60519 | 1326_at | Caspase 10 | UP |
| U66838 | 1914_at | Cyclin A | DOWN |
| U83598 | 1331_s_at | LARD (DR3) | UP |
| U91616 | 38276_at | IkB epsilon | UP |
| X04571 | 1542_at | epidermal Growth Factor | DOWN |
| X15882 | 34802_at | Collagen alpha2 typeVI | DOWN |
| X52560 | 38354_at | NF-IL6 | DOWN |
| X94216 | 1934_s_at | VEGF-C | DOWN |
| Y00272 | 40915_r_at | CDC2 | DOWN |

TABLE 1f

| | | | CP CyT |
|---|---|---|---|
| | | | fold |
| Set | Accession | Gene Information | change |
| 39473_r_at | W29065 | Cluster Incl. W29065: 56g2 Homo sapiens cDNA /gb = W29065 /gi = 1309094 /ug = Hs.110820 /len = 916 | −5.02723 |
| 34410_at | U49260 | Cluster Incl. U49260: Human mevalonate pyrophosphate decarboxylase (MPD) mRNA, complete cds | −4.425488 |
| | | /cds = (7, 1209) /gb = U49260 /gi = 1235681 /ug = Hs.3828 /len = 1795 | |
| 1089_i_at | M64936 | M64936 /FEATURE = /DEFINITION = HUMRIRT Homo sapiens retinoic | −3.824298 |
| | | acid-inducible endogenous retroviral DNA | |
| 39339_at | AB018335 | Cluster Incl. AB018335: Homo sapiens mRNA for KIAA0792 protein, complete cds /cds = (250, 2673) | −2.865299 |
| | | /gb = AB018335 /gi = 3882304 /ug = Hs.119387 /len = 4074 | |
| 32794_g_at | X00437 | Cluster Incl. X00437: Human mRNA for T-cell specific protein /cds = (37, 975) /gb = X00437 /gi = 36748 | −2.853863 |
| | | /ug = Hs.2003 /len = 1151 | |
| 40635_at | AF089750 | Cluster Incl. AF089750: Homo sapiens flotillin-1 mRNA, complete cds /cds = (164, 1447) /gb = AF089750 | −2.843357 |
| | | /gi = 3599572 /ug = Hs.179986 /len = 1796 | |
| 34293_at | AF004426 | Cluster Incl. AF004426: Homo sapiens microtubule-based motor (HsKIFC3) mRNA, complete | −2.821037 |
| | | cds /cds = (0, 2063) /gb = AF004426 /gi = 3249734 /ug = Hs.23131 /len = 2064 | |
| 36485_at | U85647 | Cluster Incl. U85647: Homo sapiens small optic lobes homolog (SOLH) mRNA, complete cds /cds = | −2.793421 |
| | | (363, 3623) /gb = U85647 /gi = 3462350 /ug = Hs.55836 /len = 4163 | |
| 41270_at | AA019936 | Cluster Incl. AA019936: ze63h04.s1 Homo sapiens cDNA, 3 end /clone = IMAGE-363703 /clone_end = 3 | −2.601016 |
| | | /gb = AA019936 /gi = 1483743 /ug = Hs.228131 /len = 538 | |
| 35473_at | Z74615 | Cluster Incl. Z74615: H. sapiens mRNA for prepro-alpha1(I) collagen /cds = (119, 4513) /gb = Z74615 | −2.45137 |
| | | /gi = 1418927 /ug = Hs.172928 /len = 6728 | |
| 40274_at | U48213 | Cluster Incl. U48213: Human D-site binding protein gene, promoter region and /cds = (375, 1352) | −2.370751 |
| | | /gb = U48213 /gi = 1245166 /ug = Hs.155402 /len = 1626 | |
| 1369_s_at | M28130 | M28130 /FEATURE = mRNA /DEFINITION = HUMIL8A Human interleukin 8 (IL8) gene, complete cds | −2.366459 |
| 32625_at | X15357 | Cluster Incl. X15357: Human mRNA for natriuretic peptide receptor (ANP-A receptor) /cds = (43, 3228) | −2.339088 |
| | | /gb = X15357 /gi = 28229 /ug = Hs.167382 /len = 3803 | |
| 36193_at | U52522 | Cluster Incl. U52522: Human arfaptin 2, putative target protein of ADP-ribosylation factor, mRNA, | −2.334116 |
| | | complete cds /cds = (67, 1092) /gb = U52522 /gi = 1279762 /ug = Hs.75139 /len = 1654 | |
| 349_g_at | D14678 | D14678 /FEATURE = /DEFINITION = HUMMHCB Human mRNA for | −2.327263 |
| | | kinesin-related protein, partial cds | |
| 38845_at | R89044 | Cluster Incl. R89044: ym99b08.s1 Homo sapiens cDNA, 3 end /clone = IMAGE-167031 /clone_end = 3 | −2.320267 |
| | | /gb = R89044 /gi = 953871 /ug = Hs.92261 /len = 477 | |
| 41074_at | AF062006 | Cluster Incl. AF062006: Homo sapiens orphan G protein-coupled receptor HG38 mRNA, complete | −2.306947 |
| | | cds /cds = (48, 2771) /gb = AF062006 /gi = 3366801 /ug = Hs.98384 /len = 2880 | |
| 408_at | X54489 | X54489 /FEATURE = mRNA /DEFINITION = HSMGSAG Human gene for melanoma growth | −2.230037 |
| | | stimulatory activity (MGSA) | |
| 36530_g_at | AI885381 | Cluster Incl. AI885381: wl93b01.x1 Homo sapiens cDNA, 3 end /clone = IMAGE-2432425 /clone_end = | −2.20277 |
| | | 3 /gb = AI885381 /gi = 5590545 /ug = Hs.61273 /len = 668 | |
| 36727_at | M64936 | Cluster Incl. M64936: Homo sapiens retinoic acid-inducible endogenous retroviral DNA /cds = UNKNOWN | −2.11194 |
| | | /gb = M64936 /gi = 337422 /ug = Hs.55322 /len = 3307 | |
| 38524_at | U49184 | Cluster Incl. U49184: Human occludin mRNA, complete cds /cds = (167, 1735) /gb = U49184 /gi = 1276978 | −2.097408 |
| | | /ug = Hs.171952 /len = 2369 | |
| 41711_at | AB019694 | Cluster Incl. AB019694: Homo sapiens mRNA for thioredoxin reductase II alpha, partial cds /cds = (0, 1574) | −2.086154 |
| | | /gb = AB019694 /gi = 4827176 /ug = Hs.12971 /len = 1931 | |
| 32964_at | X81479 | Cluster Incl. X81479: H. sapiens mRNA for EMR1 hormone receptor /cds = (38, 2698) /gb = X81479 | −2.0007 |
| | | /gi = 784993 /ug = Hs.2375 /len = 3118 | |
| 37898_r_at | AI985964 | Cluster Incl. AI985964: wr79d08.x1 Homo sapiens cDNA, 3 end /clone = IMAGE-2493903 /clone_end = | 2.051241 |
| | | 3 /gb = AI985964 /gi = 5813241 /ug = Hs.82961 /len = 487 | |
| 39544_at | AB002351 | Cluster Incl. AB002351: Human mRNA for KIAA0353 gene, partial cds /cds = (0, 4125) /gb = AB002351 | 2.066011 |
| | | /gi = 2224646 /ug = Hs.10587 /len = 6651 | |
| 538_at | S53911 | S53911 /FEATURE = /DEFINITION = S53911 CD34 = glycoprotein expressed in | 2.099395 |
| | | lymphohematopoietic progenitor cells | |
| | | {alternatively spliced, truncated form} [human, UT7, mRNA, 2657 nt] | |
| 38747_at | M81945 | Cluster Incl. M81945: Human CD34 gene, promoter and /cds = (258, 1415) /gb = M81945 /gi = 409018 | 2.133634 |
| | | /ug = Hs.85289 /len = 2616 | |
| 31834_r_at | AB020644 | Cluster Incl. AB020644: Homo sapiens mRNA for KIAA0837 protein, partial cds /cds = (0, 2237) | 2.168826 |
| | | /gb = AB020644 /gi = 4240162 /ug = Hs.14945 /len = 4868 | |
| 34235_at | AB018301 | Cluster Incl. AB018301: Homo sapiens mRNA for KIAA0758 protein, partial cds /cds = (0, 2961) /gb = | 2.185644 |
| | | AB018301 /gi = 3882236 /ug = Hs.22039 /len = 4353 | |
| 37187_at | M36820 | Cluster Incl. M36820: Human cytokine (GRO-beta) mRNA, complete cds /cds = (74, 397) /gb = M36820 | 2.250091 |
| | | /gi = 183628 /ug = Hs.75765 /len = 1110 | |
| 753_at | D86425 | D86425 /FEATURE = /DEFINITION = D86425 Homo sapiens mRNA for | 2.255818 |
| | | osteonidogen, complete cds | |
| 599_at | M60721 | M60721 /FEATURE = mRNA /DEFINITION = HUMHB24 Human homeobox gene, complete cds | 2.283385 |
| 33358_at | W29087 | Cluster Incl. W29087: 56b8 Homo sapiens cDNA /gb = W29087 /gi = 1309053 | 2.306305 |
| | | /ug = Hs.21894 /len = 877 | |
| 39039_s_at | AI557497 | Cluster Incl. AI557497: Pt2.1_16_A04.r Homo sapiens cDNA, 3 end /clone_end = 3 | 2.314698 |
| | | /gb = AI557497 /gi = 4489860 /ug = Hs.11498 /len = 862 | |
| 34262_at | Y15909 | Cluster Incl. Y15909: Homo sapiens mRNA for dia-156 protein /cds = (350, 3655) /gb = Y15909 | 2.387655 |
| | | /gi = 3171905 /ug = Hs.226483 /len = 9347 | |
| 37539_at | AB023176 | Cluster Incl. AB023176: Homo sapiens mRNA for KIAA0959 protein, partial cds /cds = (0, 2463) | 2.388291 |
| | | /gb = AB023176 /gi = 4589561 /ug = Hs.79219 /len = 4703 | |
| 37872_at | AF072468 | Cluster Incl. AF072468: Homo sapiens (JH8) mRNA, partial cds /cds = (0, 1251) /gb = AF072468 | 2.411623 |
| | | /gi = 3435202 /ug = Hs.142296 /len = 1700 | |
| 34495_r_at | AJ011733 | Cluster Incl. AJ011733: Homo sapiens mRNA for synaptogyrin 4 protein /cds = (109, 813) | 2.665967 |
| | | /gb = AJ011733 /gi = 4128018 /ug = Hs.120857 /len = 872 | |
| 37013_at | X16295 | Cluster Incl. X16295: Human mRNA for angiotensin I converting enzyme (ACE) /cds = (28, 2226) | 2.666618 |
| | | /gb = X16295 /gi = 28264 /ug = Hs.76368 /len = 2477 | |
| 34832_s_at | AB018306 | Cluster Incl. AB018306: Homo sapiens mRNA for KIAA0763 protein, complete cds | 2.760503 |
| | | /cds = (106, 2631) /gb = AB018306 /gi = 3882246 /ug = Hs.4764 /len = 4148 | |
| 40352_at | AF060862 | Cluster Incl. AF060862: Homo sapiens unknown mRNA /cds = (84, 443) /gb = AF060862 | 2.852279 |
| | | /gi = 3094013 /ug = Hs.71791 /len = 711 | |
| 32168_s_at | U85267 | Cluster Incl. U85267: Homo sapiens down syndrome candidate region 1 (DSCR1) gene, | 2.869839 |
| | | alternative exon 1, complete cds /cds = (84, 677) /gb = U85267 | |
| | | /gi = 2612867 /ug = Hs.184222 /len = 2272 | |
| 33534_at | X89426 | Cluster Incl. X89426: H. sapiens mRNA for ESM-1 protein /cds = (55, 609) /gb = X89426 /gi = 1150418 | 3.296108 |
| | | /ug = Hs.41716 /len = 2006 | |
| 34598_at | X98085 | Cluster Incl. X98085: H. sapiens mRNA for tenascin-R /cds = (117, 4193) /gb = X98085 /gi = 1617315 | 3.647829 |
| | | /ug = Hs.54433 /len = 4738 | |
| 33803_at | J02973 | Cluster Incl. J02973: Human thrombomodulin gene, complete cds /cds = (541, 2268) /gb = J02973 | 3.757156 |
| | | /gi = 339658 /ug = Hs.2030 /len = 4050 | |
| 37710_at | L08895 | Cluster Incl. L08895: Homo sapiens MADS/MEF2-family transcription factor (MEF2C) mRNA, complete cds | 3.965913 |
| | | /cds = (401, 1822) /gb = L08895 /gi = 292289 /ug = Hs.78995 /len = 4077 | |
| 31740_s_at | AB008913 | Cluster Incl. AB008913: Homo sapiens mRNA for Pax-4, complete cds /cds = (0, 1052) /gb = AB008913 | 4.282782 |
| | | /gi = 2809074 /ug = Hs.129706 /len = 1088 | |
| 40223_r_at | AI677689 | Cluster Incl. AI677689: wd33c06.x1 Homo sapiens cDNA, 3 end /clone = IMAGE-2329930 /clone_end = | 6.492528 |
| | | 3 /gb = AI677689 /gi = 4887871 /ug = Hs.153121 /len = 478 | |

TABLE 2
Abundant endothelial transcripts

| Transcript | Abundance | Probe set |
|---|---|---|
| G-protein signaling | | |
| G-protein alpha subunit S | 85.8 | 37449_i_at |
| RACK1 | 122.6 | 34608_at |
| Carbohydrate metabolism | | |
| aldolase A | 91.8 | 32336_at |
| phosphoglycerate mutase 1 | 87.2 | 41221_at |
| GAPDH | 138.3 | M33197_3_at |
| Cytoskeleton | | |
| beta-tubulin | 107.4 | 151_s_at |
| thymosin beta-4 | 119.7 | 31557_at |
| myosin light chain | 87.8 | 33994_g_at |
| vimentin | 132.4 | 34091_s_at |
| gamma actin 1 | 117.5 | 34160_at |
| beta-actin | 164.6 | X00351_M_at |
| Ribosomal proteins | | |
| ribosomal protein S3A | 83.8 | 1653_at |
| ribosomal protein L10 | 118.8 | 2016_s_at |
| ribosomal protein S19 | 93.5 | 31330_at |
| ribosomal protein L28 | 100.9 | 31385_at |
| ribosomal protein L8 | 99.9 | 31505_at |
| ribosomal protein S2 | 125.2 | 31527_at |
| ribosomal protein S18 | 97.3 | 31545_at |
| ribosomal protein S10 | 83.6 | 31568_at |
| ribosomal Protein L3 | 93.6 | 31722_at |
| ribosomal phosphoprotein P1 | 90.3 | 31956_f_at |
| ribosomal phosphoprotein P1 | 111.1 | 31957_r_at |
| ribosomal protein L37a | 125.8 | 31962_at |
| ribosomal protein L32 | 81.7 | 32276_at |
| ribosomal protein S11 | 87.7 | 32330_at |
| ribosomal protein S14 | 93.4 | 32412_at |
| ribosomal protein S5 | 87.2 | 32437_at |
| ribosomal protein S20 | 121.4 | 32438_at |
| ribosomal protein L41 | 121.9 | 32466_at |
| ribosomal protein S21 | 84.6 | 32744_at |
| ribosomal protein S12 | 99.6 | 33116_f_at |
| ribosomal protein L38 | 98.3 | 34085_at |
| ribosomal protein S17 | 109.0 | 34592_at |
| ribosomal protein S17 | 104.0 | 34593_g_at |
| ribosomal protein S4 | 90.2 | 34643_at |
| ribosomal protein S3 | 108.0 | 34645_at |
| ribosomal protein S28 | 99.0 | 347_s_at |
| ribosomal protein L13a | 84.9 | 35119_at |
| Miscelaneous | | |
| laminin receptor (non-integrin) | 128.6 | 256_s_at |
| Annexin A2 (lipocortin II) | 171.8 | 769_s_at |
| MIF | 90.2 | 895_at |
| plasminogen activator inhibitor I | 111.4 | 38125_at |
| elongation factor 1-alpha | 148.6 | 1288_s_at |
| ubiquitin C | 91.5 | 1367_f_at |
| enolase 1 | 93.6 | 2035_s_at |
| polyubiquitin UbC | 97.9 | 32334_f_at |
| benzodiazepine receptor | 88.2 | 32806_at |
| cyclophilin A | 97.0 | 33667_at |
| elongation factor 1-alpha | 144.0 | 40887_g_at |
| ESTs | | |
| EST AI535946 | 114.5 | 33412_at |
| EST AI541542 | 113.8 | 35278_at |
| EST U34995 | 172.4 | 35905_s_at |

TABLE 3
Endothelial-biased transcripts

| Transcript | Et/BL | Et/Em | Probe set |
|---|---|---|---|
| transcription | | | |
| HHEX (homeobox) | 14 | 14 | 37497_at |
| erg | 265 | 22 | 914_g_at |
| adhesion/matrix | | | |
| integrin alpha 6B | 24 | 11 | 33410_at |
| VE-cadherin | 110 | 44 | 37196_at |
| PECAM-1 (CD31) | 77 | 17 | 37398_at |
| MMP I | 419 | 757 | 38428_at |
| integrin alpha 5 | 57 | 13 | 39753_at |
| growth factors | | | |
| TSG-14 | 404 | 2294 | 1491_at |
| VEGF-C | 17 | 12 | 1934_s_at |
| IGF BP 10 | 250 | 14 | 38772_at |
| BMP-6 | 87 | 12 | 39279_at |
| angiopoietin-2 | 32 | 43 | 37461_at |
| PIGF | 37 | 105 | 793_at |
| recpetors | | | |
| Eph-A4 | 12 | 18 | 1606_at |
| TGF-beta RII | 92 | 10 | 1814_at |
| PECAM-1 | 133 | 61 | 268_at |
| TMP | 58 | 12 | 37762_at |
| IL1 receptor 1 | 27 | 12 | 40322_at |
| p27 | 24 | 13 | 425_at |
| miscellaneous | | | |
| ras inhibitor SF4 | 11 | 36 | 1783_at |
| IPL | 27 | 22 | 31888_s_at |
| solute carrier 16 | 82 | 14 | 33143_s_at |
| endothelial-specific-1 | 222 | 344 | 33534_at |
| RGS 5 | 62 | 11 | 33890_at |
| PLOD2 | 38 | 10 | 34795_at |
| filamin C | 23 | 17 | 35330_at |
| myosin X | 129 | 10 | 35362_at |
| SCHIP-1 | 30 | 22 | 36536_at |
| ribonuclease A | 60 | 15 | 37402_at |
| HERMES | 16 | 84 | 38049_g_at |
| PAI-I | 187 | 52 | 38125_at |
| trypsinogen IV | 16 | 12 | 40043_at |
| serine protease SIG13 | 347 | 10 | 40078_at |
| MAP 5 | 13 | 11 | 41373_s_at |
| Von Willebrand factor | 98 | 18 | 607_s_at |
| ESTs | | | |
| EST AL080215 | 13 | 11 | 32454_at |
| EST AB023155 | 16 | 11 | 33235_at |
| EST AI672098 | 10 | 60 | 33407_at |
| EST AB007889 | 23 | 61 | 37363_at |
| EST Y09836 | 26 | 11 | 38396_at |
| EST AB014520 | 17 | 23 | 38671_at |
| EST AF000959 | 87 | 29 | 38995_at |
| EST AI743090 | 14 | 16 | 39549_at |
| EST AF001436 | 10 | 25 | 41658_at |

1. A method of monitoring the progression of a disease condition associated with angiogenesis or vassculogenesis in a human subject, said method comprising:
making a quantitative determination of the transcript level of at least one gene shown in table 1 in a sample comprising cells obtained from the site of said disease; and
comparing the transcript level so determined with the transcript level of at least one gene obtained from a control sample of cells.

2. The method of claim 1 wherein said control sample is obtained from the disease site of said patient at an earlier point in time.

3. The method of claim 1 wherein said control sample is obtained from endothelial cells in non-diseased tissue in said patient.

4. The method of claim 1, wherein said determination is made after a course of treatment of said patient.

5. The method of claim 1 wherein the transcript level is determined for at least one transcription regulator; at least one apoptosis regulator, at least one growth factor or growth factor receptor, and at least one adhesion/matrix protein.

6. The method of claim 1 wherein the transcript level of at least 5 genes is determined.

7. The method of claim 6 wherein the transcript level of at least 10 genes is determined.

8. The method of claim 1 wherein the transcript level is determined for at least one gene of table 1 a.

9. The method of claim 1 wherein the transcript level is determined by hybridization to a gene chip array.

10. The method of claim 1 wherein the transcript level is determined by quantitative PCR.

11. The method of claim 1 wherein said disease condition is a disease associated with unwanted cellular proliferation, including solid tumors.

12. The method of claim 1 wherein the disease condition is associated with a lack of vasculature.

13. A gene chip array suitable for use in the method of claim 1 comprising at least one nucleic acid suitable for detection of at least one gene shown in Table 1; optionally a control specific for said at least one gene; and optionally at least one control for said gene chip.

14. An assay method for a modulator of angiogenesis or vasculogenesis, wherein said method comprises:
(a) providing a protein selected from Table 1;
(b) bringing said protein into contact with a candidate modulator of its activity; and
(c) determining whether said candidate modulator is capable of modulating the activity of said protein.

15. An assay method according to claim 14 wherein said candidate modulator is an antibody or binding fragment thereof which binds said protein.

16. An assay method according to claim 14 wherein said candidate modulator is a fragment of said protein or mimetic thereof.

17. An assay method for a modulator of angiogenesis or vasculogenesis, wherein said method comprises;
(a) providing an endothelial cell in culture;
(b) bringing said cell into contact with a candidate modulator of angiogenesis; and
(c) determining whether said candidate modulator is capable of modulating the transcription of at least one gene selected from the genes of Table 1.

18. An assay method according to claim 17 wherein said candidate modulator is an antisense oligonucleotide.

19. Use of a modulator obtained from the assay method of claim 14 in a method of modulating angiogenesis or vasculogenesis in a human patient.

20. A vector comprising an EST sequence from Table 1 operably linked to a promoter for transcription of said sequence.

21. The vector of claim 20 wherein said EST sequence is linked in-frame for to a translational initiation region for translation of said sequence.

22. The vector of claim 20 wherein said EST sequence is in an anti-sense orientation.
